(19) United States
(12) Patent Application Publication
(10) Pub. No.: US 2015/0238091 A1
(43) Pub. Date: Aug. 27, 2015

(54) PHOTOACOUSTIC MONITORING TECHNIQUE WITH NOISE REDUCTION

(71) Applicant: Covidien LP, Mansfield, MA (US)

(72) Inventors: Darshan Iyer, Superior, CO (US); William Kit Dean, Castle Pines, CO (US); Youzhi Li, Longmont, CO (US)

(21) Appl. No.: 14/607,302

(22) Filed: Jan. 28, 2015

Related U.S. Application Data
(60) Provisional application No. 61/943,612, filed on Feb. 24, 2014.

(51) Int. Cl. A61B 5/00; A61B 5/029

(52) U.S. Cl. CPC A61B 5/0095; A61B 5/489; A61B 5/0044; A61B 5/7278; A61B 5/7203; A61B 5/725; A61B 5/726; A61B 5/7228; A61B 5/029; A61B 2576/023

(57) ABSTRACT

Various methods and systems for photoacoustic patient monitoring are provided. A photoacoustic system includes a light emitting component that emits one or more wavelengths of light into an interrogation region of a patient and an acoustic detector that detects acoustic energy generated by the interrogation region of the patient in response to the emitted light. The system also includes techniques to remove noise from the signal generated by the acoustic detector.

### CROSS-REFERENCE TO RELATED APPLICATION

This application claims priority to and the benefit of U.S. Provisional Application No. 61/943,612, entitled “PHOTOACOUSTIC MONITORING TECHNIQUE WITH NOISE REDUCTION”, filed Feb. 24, 2014, which is herein incorporated by reference in its entirety.

### BACKGROUND

The present disclosure relates generally to medical devices and, more particularly, to the use of photoacoustic spectroscopy in patient monitoring.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring patient characteristics. Such devices provide doctors and other healthcare personnel with the information they need to provide healthcare for their patients. As a result, such monitoring devices have become an indispensable part of modern medicine. For example, clinicians may wish to monitor a patient's blood flow to assess cardiac function. In particular, clinicians may wish to monitor a patient's cardiac output. The determination of cardiac output may provide information useful for the diagnosis and treatment of various disease states or patient abnormalities. For example, in cases of pulmonary hypertension, a clinical response may include a decrease in cardiac output.

Accordingly, there are a variety of clinical techniques that may be used for analyzing cardiac output. In one technique, an indicator, such as a dye or saline solution, is injected into a circulatory system of a patient, and information about certain hemodynamic parameters may be determined by assessing the dilution of the indicator after mixing with the bloodstream. However, such techniques involve invasive artery catheters for detecting the dilution of the indicator. Other techniques may involve radioactive indicators that are easier to detect, but expose the patient to radioactivity and involve expensive detection equipment.

### BRIEF DESCRIPTION

Provided herein are non-invasive photoacoustic techniques that are capable of measuring indicator dilution. For example, for patients with an indicator solution injected into a vein, photoacoustic monitoring techniques may be used to measure dilution of the indicator in a downstream artery after mixing in the blood. The extent of dilution relates to cardiac output and other hemodynamic parameters. Such techniques may involve a photoacoustic sensor and/or an associated monitoring system or methods used in conjunction with such sensors and/or systems.

The disclosed embodiments include a monitor. The monitor includes a memory that stores instructions for: receiving a signal from an acoustic detector configured to detect a photoacoustic effect from light emitted into a patient's tissue, wherein the signal is representative of an indicator dilution, dividing the signal into a plurality of blocks, wherein each block includes a same number of epochs, arranging the respective epochs within each block of the plurality of blocks into an ensemble arrangement, and identifying a region of each respective epoch with each block that corresponds to a blood vessel response. The memory also stores instructions for: calculating an indicator dilution curve based on the identified regions of the epochs, identifying a signal region of the indicator dilution curve, extracting one or more features from the signal region of the indicator dilution curve, and determining a physiological parameter based on the one or more features. The monitor also includes a processor configured to execute the instructions.

The disclosed embodiments also include a method for determining a physiological parameter of a patient performed using a processor. The method includes the steps of receiving a signal from an acoustic detector configured to detect a photoacoustic effect from light emitted into a patient's tissue, wherein the signal is representative of an indicator dilution, dividing the signal into a plurality of blocks, wherein each block includes a same number of epochs, arranging the respective epochs within each block into an ensemble arrangement, and identifying a region of each respective epoch that corresponds to a blood vessel response utilizing an ensemble-averaged envelope calculated for each block of the plurality of blocks based on the respective epochs within each block. The method also includes the steps of calculating an indicator dilution curve based on the identified regions of the epochs, identifying a signal region of the indicator dilution curve, extracting one or more features from the signal region of the indicator dilution curve, and determining a physiological parameter based on the one or more features, such as by utilizing ridge regression.

The disclosed embodiments also include non-transitory computer-readable medium having computer executable code stored thereon. The codes includes instructions for: receiving a signal from an acoustic detector configured to detect a photoacoustic effect from light emitted into a patient's tissue, wherein the signal is representative of an indicator dilution, dividing the signal into a plurality of blocks, wherein each block includes a same number of epochs, and identifying a region of each respective epoch within each block that corresponds to a blood vessel response. The code also includes instructions for: calculating an indicator dilution curve based on the identified regions of the epochs, identifying a signal region of the indicator dilution curve, extracting one or more features from the signal region of the indicator dilution curve, and determining a physiological parameter based on the one or more features, such as by utilizing ridge regression.

### BRIEF DESCRIPTION OF THE DRAWINGS

Advantages of the disclosed techniques may become apparent upon reading the following detailed description and upon reference to the drawings in which:

FIG. 1 is a block diagram of a patient monitor and photoacoustic sensor in accordance with an embodiment;

FIG. 2 is a flow diagram of a method of determining a physiological parameter (e.g., hemodynamic parameter) in accordance with an embodiment;

FIG. 3 is a flow diagram of a method of determining a (e.g., hemodynamic parameter) hemodynamic parameter in accordance with an embodiment;

FIG. 4 is a graphical representation of a portion of the normalized indicator dilution (ID) curve illustrating features that may extracted from the ID curve;

FIG. 5 is a flow diagram of a method of utilizing a k-means clustering-based approach to calculate an average photoacoustic (PA) signal from a block of raw PA data in accordance with an embodiment;

FIG. 6 is a graphical representation of arranging raw PA data into an ensemble of PA epochs;

FIG. 7 is a graphical representation of utilizing a k-means clustering-based approach of FIG. 5 to calculate an average PA signal for the ensemble of PA epochs in FIG. 6;

FIG. 8 is a flow diagram of a method of utilizing a Woody filtering based approach to calculate an average PA signal from a block of raw PA data in accordance with an embodiment;

FIG. 9 is a graphical representation of utilizing the Woody filtering based approach of FIG. 8 to calculate an average PA signal for the ensemble of PA epochs in FIG. 6;

FIG. 10 is a flow diagram of a method of utilizing a correlation-based approach to calculate an average PA signal from a block of raw PA data in accordance with an embodiment;

FIG. 11 is a graphical representation of utilizing the correlation-based approach of FIG. 10 to calculate the average PA signal for the ensemble of PA epochs in FIG. 6;

FIG. 12 is a flow diagram of a method of utilizing a wavelet-based approach to denoise a PA signal in accordance with an embodiment;

FIG. 13 is a flow diagram of a method of utilizing a complex demodulation-based approach to extract PA data from a PA signal corresponding to a blood vessel in accordance with an embodiment;

FIG. 14 is graphical representation of utilizing the complex demodulation-based approach of FIG. 12 to isolate a region of the PA signal corresponding to the blood vessel;

FIG. 15 is a flow diagram of a method of utilizing a complex demodulation-based approach to calculate a raw ID curve in accordance with an embodiment;

FIG. 16 is a graphical representation of a raw PA epoch, an enveloped derived from the raw PA epoch utilizing the complex demodulation-based approach of FIG. 15, and a region of the envelope corresponding to a blood vessel;

FIG. 17 is a graphical representation of a raw ID curve calculated utilizing the method of FIG. 15 and a filtered ID curve of the raw ID curve;

FIG. 18 is a flow diagram of a method of utilizing a power signal to calculate a raw ID curve in accordance with an embodiment;

FIG. 19 is a graphical representation of a raw PA epoch, a power signal derived from the raw PA epoch utilizing the method of FIG. 18, and a region of the power signal corresponding to a blood vessel;

FIG. 20 is a graphical representation of a raw ID curve calculated utilizing the method of FIG. 18 and a filtered ID curve of the raw ID curve;

FIG. 21 is a flow diagram of a method of utilizing adaptive filter selection to denoise a raw ID curve in accordance with an embodiment;

FIG. 22 is a graphical representation of a raw ID curve and a filtered ID curve from a single specific filter;

FIG. 23 is a graphical representation of demarcation of signal and noise regions of a filtered ID curve;

FIG. 24 is a graphical representation of the signal-to-noise ratio (SNR) for a plurality of filters with different cutoffs for a specific ID curve;

FIG. 25 is a flow diagram of a method of utilizing an independent component analysis (ICA)-based approach for denoising ID curves in accordance with an embodiment;

FIG. 26 is a graphical representation of independent components (ICs) derived from an ID curve;

FIG. 27 is a graphical representation of an ID curve derived from a sensor prior to and after ICA denoising via the method of FIG. 25;

FIG. 28 is a flow diagram of a method of selecting independent components (ICs) obtained with the method of FIG. 25 for denoising the ID curves in accordance with an embodiment;

FIG. 29 is a graphical representation of a raw ID curve and denoised ID curves derived from the raw ID curve utilizing a total variation minimization-based approach;

FIG. 30 is a flow diagram of a method of calculating the signal region of an ID curve in accordance with an embodiment;

FIG. 31 is a graphical representation of features of an ID curve used in normalization;

FIG. 32 is a graphical representation of the performance of different normalization techniques on estimating cardiac output;

FIG. 33 is a flow diagram of a method of estimating cardiac output utilizing ridge regression in accordance with an embodiment;

FIG. 34A is a graphical representation of a scatter plot for cardiac output estimates derived using an equation based-approach against actual cardiac output measurements;

FIG. 34B is a graphical representation of a scatter plot for cardiac output estimates derived using the method of FIG. 33 against actual cardiac output measurements;

FIG. 34C is a graphical representation of a Bland-Altman plot for cardiac output estimates derived using an equation based-approach against actual cardiac output measurements;

FIG. 34D is a graphical representation of a Bland-Altman plot for cardiac output estimates derived using the method of FIG. 33 against actual cardiac output measurements; and

FIG. 35 is a flow diagram of a method of determining a physiological parameter (e.g., hemodynamic parameter) utilizing a wavelet-based approach in accordance with an embodiment in accordance with an embodiment; and

FIG. 36 is a graphical representation of utilizing the method of FIG. 35 to generate ID curves for determining the hemodynamic parameter.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain medical contexts it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel. In one approach, measurement of such localized parameters is achieved via photoacoustic (PA) spectroscopy. Photoacoustic spectroscopy uses light directed into a patient's tissue to generate acoustic waves that may be detected and resolved to determine localized physiological information of interest. In particular, the light energy directed into the tissue may be provided at particular wavelengths that correspond to the absorption profile of one or more blood or tissue constituents of interest. In certain embodiments, the light is emitted as pulses (i.e., pulsed photoacoustic spectroscopy), though in other embodiments the light may be emitted in a continuous manner (i.e., continuous photoacoustic spectroscopy). The light absorbed by the constituent of interest results in a proportionate increase in the kinetic energy of the constituent (i.e., the constituent is heated), which results in the generation of acoustic waves. The acoustic waves may be detected and used to determine the amount of light absorption, and thus the quantity of the constituent of interest, in the illuminated region. For example, the detected acoustic energy may be proportional to the optical absorption coefficient of the blood or tissue constituent and the fluence of light at the wavelength of interest at the localized region being interrogated (e.g., a specific blood vessel).

In an embodiment, an indicator dilution (ID) method is used to determine cardiac output of a patient. When an indicator is injected into a vein in a cardiovascular system, a diluted temporal profile of the indicator may be measured in a downstream artery. Measurement of this diluted temporal profile may be used to estimate the hemodynamic properties. As provided herein, the dilution measurement may be performed with a photoacoustic monitoring technique that, in particular embodiments, does not use an invasive artery catheter to capture the dilution of the indicator in the artery. Algorithms applied to photoacoustic ID curves may be used to estimate hemodynamic properties, such as cardiac output. Notably, in cases where the ID curves contain noise, such noise may come from multiple sources including patient respiration and/or perfusion and may be complex to remove with signal processing techniques due to overlapping frequency ranges. Further, light is modulated in the tissue due to cardiac cycles and respiration, resulting in changes in the amount of the photoacoustic effect over the course of the ID measurement.

The present embodiments use a photoacoustic optical light source to generate acoustic waves and various techniques to minimize noise within the detected acoustic signal and to improve the signal-to-noise ratio (SNR) of the ID curves. Minimizing the noise and improving the SNR of the ID curves may increase the accuracy of physiological parameters (e.g., cardiac output) calculated from the detected acoustic signal.

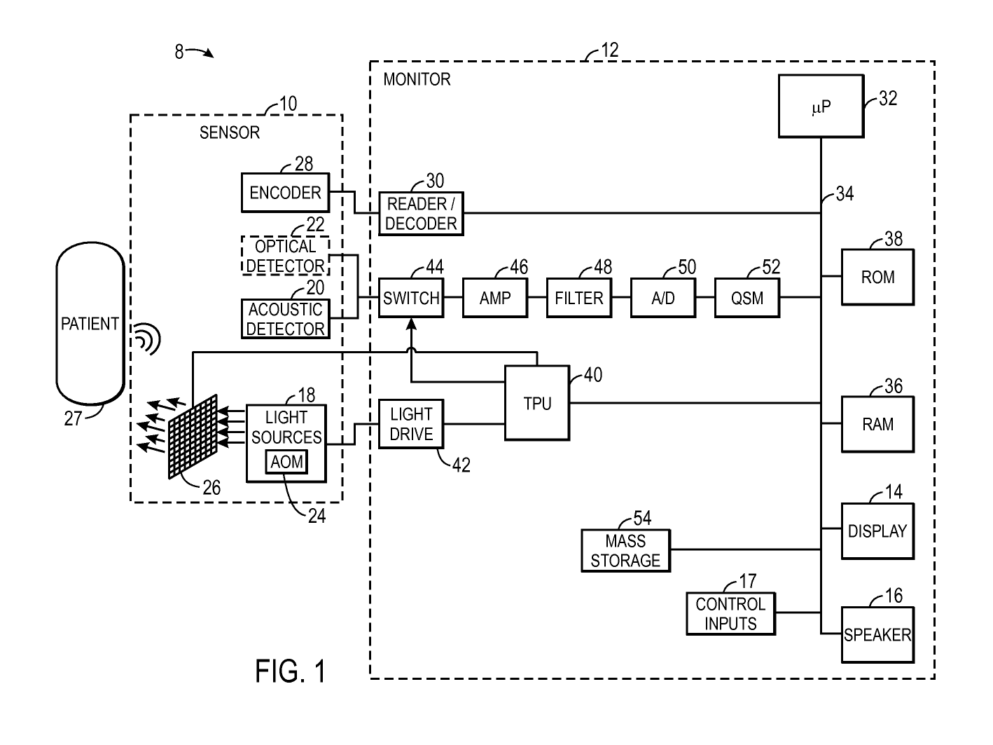

With the foregoing in mind, FIG. 1 depicts an example of a photoacoustic monitoring system 8 that may be utilized in determining cardiac output. The system 8 includes a photoacoustic sensor 10 and a monitor 12. Some photoacoustic systems 8 may include one or more photoacoustic sensors 10, as illustrated in FIG. 1, to generate physiological signals for different regions of a patient. For example, in certain embodiments, a single sensor 10 may have sufficient penetration depth to generate physiological signals from deep vessels (e.g., pulmonary artery and/or pulmonary vein). In other embodiments, more than one (e.g., two sensors) sensor 10 may be used to monitor physiological parameters (e.g., oxygen saturation) of more superficial vessels (e.g., the jugular vein and the femoral vein). Further, a system 8 as contemplated may be used in conjunction with other types of medical sensors, e.g., pulse oximetry or regional saturation sensors, to provide input to a multiparameter monitor. In certain embodiments, the photoacoustic sensor 10 may be wireless (i.e., communicate wirelessly with the monitor 12).

The sensor 10 may emit light at certain wavelengths into a patient's tissue and may detect acoustic waves (e.g., ultrasound waves) generated in response to the emitted light. The monitor 12 may be capable of calculating physiological characteristics based on signals received from the sensor 10 that correspond to the detected acoustic waves. The monitor 12 may include a display 14 and/or a speaker 16 which may be used to convey information about the calculated physiological characteristics to a user. Further, the monitor 12 may be configured to receive user inputs via control input circuitry 17. The sensor 10 may be communicatively coupled to the monitor 12 via a cable or, in some embodiments, via a wireless communication link.

In one embodiment, the sensor 10 may include a light source 18 and an acoustic detector 20, such as an ultrasound transducer. The disclosed embodiments may generally describe the use of continuous wave (CW) light sources to facilitate explanation. However, it should be appreciated that the photoacoustic sensor 10 may also be adapted for use with other types of light sources, such as pulsed light sources, in other embodiments. In certain embodiments, the light source 18 may be associated with one or more optical fibers for conveying light from one or more light generating components to the tissue site. In certain embodiments, the sensor 10 also includes an optical detector 22 that may be a photodetector, such as a silicon photodiode package, selected to receive light in the range emitted from the light source 18. In the present context, the optical detector 22 may be referred to as a detector, a photodetector, a detector device, a detector assembly or a detector component. Further, the detector 22 and light source 18 may be referred to as optical components or devices. In certain embodiments, the optical detector 22 may include one or more of a flat optical detector and/or a focused optical detector.

For example, in one embodiment the light source 18 may be one, two, or more light emitting components (such as light emitting diodes) adapted to transmit light at one or more specified wavelengths. In certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1100 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1100 nm) may be used. In one embodiment, the selected wavelengths of light may penetrate between 1 mm to 3 cm into the tissue of the patient. In certain embodiments, the selected wavelengths may penetrate through bone (e.g., the rib cage) of the patient.

One problem that may arise in photoacoustic spectroscopy may be attributed to the tendency of the emitted light to diffuse or scatter in the tissue of the patient. As a result, light emitted toward an internal structure or region, such as a blood vessel, may be diffused prior to reaching the region so that amount of light reaching the region is less than desired. Therefore, due to the diffusion of the light, less light may be available to be absorbed by the constituent of interest in the target region, thus reducing the acoustic waves generated at the target region of interest, such as a blood vessel. To increase the precision of the measurements, the emitted light may be focused on an internal region of interest by modulating or coding the intensity and/or phase or frequency of the illuminating light. In some embodiments, cross-illumination of a tissue field (i.e., region of interest) may occur with the modulated or coded illuminated light from separate sources 18 or sensors 10.

Accordingly, an acousto-optic modulator (AOM) 24 may modulate the intensity of the emitted light, for example, by using LFM techniques. The emitted light may be intensity modulated by the AOM 24 or by changes in the driving current of the LED emitting the light. The intensity modulation may result in any suitable frequency, such as from 1 MHz to 10 MHz or more. Accordingly, in one embodiment, the light source 18 may emit LFM chirps at a frequency sweep range approximately from 1 MHz to 5 MHz. In another embodiment, the frequency sweep range may be of approximately 0.5 MHz to 10 MHz. The frequency of the emitted light may be increasing with time during the duration of the chirp. In certain embodiments, the chirp may last approximately 0.1 second or less and have an associated energy of a 10 mJ or less, such as between 1 μJ to 2 mJ, 1-5 mJ, or 1-10 mJ. In such an embodiment, the limited duration of the light may prevent heating of the tissue while still emitting light of sufficient energy into the region of interest to generate the desired acoustic waves when absorbed by the constituent of interest.

Additionally, or alternatively, the light emitted by the light source 18 may be spatially modulated, such as via a modulator 26. For example, in one embodiment, the modulator 26 may be a spatial light modulator, such as a Holoeye® LC-R 2500 liquid crystal spatial light modulator. In one such embodiment, the spatial light modulator may have a resolution of 1024×768 pixels or any other suitable pixel resolution. During operation, the pixels of the modulator 26 may be divided into subgroups (such as square or rectangular subarrays or groupings of pixels) and the pixels within a subgroup may generally operate together. For example, the pixels of a modulator 26 may be generally divided into square arrays of 10×10, 20×20, 40×40, or 50×50 pixels. In one embodiment, each subgroup of pixels of the modulator 26 may be operated independently of the other subgroups. The pixels within a subgroup may be operated jointly (i.e., are on or off at the same time) though the subgroups themselves may be operated independently of one another. In this manner, each subgroup of pixels of the modulator 26 may be operated so as to introduce phase differences at different spatial locations within the emitted light. That is, the modulated light that has passed through one subgroup of pixels may be at one phase and that phase may be the same or different than the modulated light that has passed through other subgroups of pixels, i.e., some segments or portions of the modulated light wavefront may be ahead of or behind other portions of the wavefront. In one embodiment, the modulator 26 may be associated with additional optical components (e.g., lenses, reflectors, refraction gradients, polarizers, and so forth) through which the spatially modulated light passes before reaching the tissue of the patient 27.

In one example, the acoustic detector 20 may be one or more ultrasound transducers, such as a focused ultrasound transducer, suitable for detecting ultrasound waves emanating from the tissue in response to the emitted light and for generating a respective optical or electrical signal in response to the ultrasound waves. For example, the acoustic detector 20 may be suitable for measuring the frequency and/or amplitude of the acoustic waves, the shape of the acoustic waves, and/or the time delay associated with the acoustic waves with respect to the light emission that generated the respective waves. In certain embodiments, the acoustic detector 20 may include a flat ultrasound detector. In one embodiment an acoustic detector 20 may be an ultrasound transducer employing piezoelectric or capacitive elements to generate an electrical signal in response to acoustic energy emanating from the tissue of the patient 27, i.e., the transducer converts the acoustic energy into an electrical signal.

In certain embodiments, the light source 18 and the acoustic detector 20 may be arranged on the same side of the sensor 10 in a reflectance-type sensor arrangement. It should be understood that transmission-type arrangements are also contemplated in which the light source 18 and the acoustic detector 20 are on opposing sides of a tissue when applied to the patient 27. In embodiments that also include the optical detector 22, the detector 22 may also be located on the same side of the sensor 10 as the light source 18 or located on the opposing side of the tissue from the light source 18 when the sensor is applied to the patient 27.

In one implementation, the acoustic detector 20 may be a low finesse Fabry-Perot interferometer mounted on an optical fiber. In such an embodiment, the incident acoustic waves emanating from the probed tissue modulate the thickness of a thin polymer film. This produces a corresponding intensity modulation of light reflected from the film. Accordingly, the acoustic waves are converted to optical information, which is transmitted through the optical fiber to an upstream optical detector, which may be any suitable detector. In some embodiments, a change in phase of the detected light may be detected via an appropriate interferometry device which generates an electrical signal that may be processed by the monitor 12. The use of the thin film as the acoustic detecting surface allows high sensitivity to be achieved, even for films of micrometer or tens of micrometers in thickness. In one embodiment, the thin film may be a 0.25 mm diameter disk of 50 micrometer thickness polyethylene terepthalate with an at least partially optically reflective (e.g., 40% reflective) aluminum coating on one side and a mirror reflective coating on the other (e.g., 100% reflective) that form the mirrors of the interferometer. The optical fiber may be any suitable fiber, such as a 50 micrometer core silica multimode fiber of numerical aperture 0.1 and an outer diameter of 0.25 mm.

In the case of pulsed photoacoustic spectrometry, the system 8 may be configured to sporadically emit pulses of light from the light source 18 onto the patient 27 at random or predetermined irregular (i.e., non-uniform) intervals, such that the light source 18 is energized for a smaller amount of time than it would be conventionally. For example, pulses may be emitted at an interval in the range of every 1 to 10 milliseconds (ms), with a laser pulse length in the range of 10 to 100 ns. The average frequency is an average of the irregular intervals at which the pulses are emitted. As such, data collected by the acoustic detector 20 and/or optical detector 22 may be a set of sporadic data samples rather than a full data set (e.g., data gathered via frequent, regular emission of light for an extended length of time).

The photoacoustic sensor 10 may include a memory or other data encoding component, depicted in FIG. 1 as an encoder 28. For example, the encoder 28 may be a solid state memory, a resistor, or combination of resistors and/or memory components that may be read or decoded by the monitor 12, such as via reader/decoder 30, to provide the monitor 12 with information about the attached sensor 10. For example, the encoder 28 may encode information about the sensor 10 or its components (such as information about the light source 18 and/or the acoustic detector 20). Such encoded information may include information about the configuration or location of photoacoustic sensor 10, information about the type of lights source(s) 18 present on the sensor 10, information about the wavelengths, light wave frequencies, chirp durations, and/or light wave energies which the light source(s) 18 are capable of emitting and the properties and/or detection range of the optical detector 22 (if present), information about the nature of the acoustic detector 20, and so forth. In certain embodiments, the information also includes a reference linear frequency modulation (LFM) chirp that was used to generate the actual LFM emitted light. This information may allow the monitor 12 to select appropriate algorithms and/or calibration coefficients for calculating the patient's physiological characteristics, such as the amount or concentration of a constituent of interest in a localized region, such as a blood vessel.

In one implementation, signals from the acoustic detector 20 (and decoded data from the encoder 28, if present) and/or the optical detector 22 (if present) may be transmitted to the monitor 12. The monitor 12 may include data processing circuitry (such as one or more processors 32, application specific integrated circuits (ASICS), or so forth) coupled to an internal bus 34. Also connected to the bus 34 may be a RAM memory 36, a ROM memory 38, a speaker 16 and/or a display 14. In one embodiment, a time processing unit (TPU) 40 may provide timing control signals to light drive circuitry 42, which controls operation of the light source 18, such as to control when, for how long, and/or how frequently the light source 18 is activated, and if multiple light sources are used, the multiplexed timing for the different light sources.

The TPU 40 may also control or contribute to operation of the acoustic detector 20 and/or the optical detector 22 such that timing information for data acquired using the acoustic detector 20 and/or the optical detector 22 may be obtained. Such timing information may be used in interpreting the acoustic wave data and/or in generating physiological information of interest from such acoustic data. For example, the timing of the acoustic data acquired using the acoustic detector 20 may be associated with the light emission profile of the light source 18 during data acquisition. Likewise, in one embodiment, data acquisition by the acoustic detector 20 may be gated, such as via a switching circuit 44, to account for differing aspects of light emission. For example, operation of the switching circuit 44 may allow for separate or discrete acquisition of data that corresponds to different respective wavelengths of light emitted at different times. Similarly, the data acquired from the optical detector 22 may be gated via the switched circuit 44.

The received signal from the acoustic detector 20 and/or the optical detector 22 may be amplified (such as via amplifier 46), may be filtered (such as via filter 48), and/or may be digitized if initially analog (such as via an analog-to-digital converter 50). The digital data may be provided directly to the processor 32, may be stored in the RAM 36, and/or may be stored in a queued serial module (QSM) 52 prior to being downloaded to RAM 36 as QSM 52 fills up. In one embodiment, there may be separate, parallel paths for separate amplifiers, filters, and/or A/D converters provided for different respective light wavelengths or spectra used to generate the acoustic data. Further, while the disclosed block diagram shows the signal from the optical detector 22 and the acoustic detector 20 being supplied to the same path (e.g., a path that may include a switch 44, amplifier 46, filter 48, A/D converter 50, and/or a QSM 52), it should be understood that these signals may be received and processed on separate paths or separate channels.

The data processing circuitry, such as processor 32, may derive one or more physiological characteristics based on data generated by the photoacoustic sensor 10. For example, based at least in part upon data received from the acoustic detector 20, the processor 32 may calculate the amount or concentration of a constituent of interest in a localized region of tissue or blood using various algorithms. In one embodiment, the processor 32 may calculate one or more of cardiac output, total blood volume, extravascular lung water, intrathoracic blood volume, oxygen saturation, and/or macro and microvascular blood flow from signals obtained from a signal sensor 10. In one embodiment, the processor 32 may calculate one or more of cardiac output, blood volume, extravascular lung water, intrathoracic blood volume, systemic and pulmonary blood flow, and/or macro and microvascular blood flow from signals obtained from a signal sensor 10. In certain embodiments, these algorithms may use coefficients, which may be empirically determined, that relate the detected acoustic waves generated in response to emitted light waves at a particular wavelength or wavelengths to a given concentration or quantity of a constituent of interest within a localized region. In one embodiment, the disclosed techniques may utilize a correlation of a detected optical signal to the detected acoustic signal to remove noise as disclosed in U.S. patent application Ser. No. 13/836,531, entitled “PHOTOACOUSTIC MONITORING TECHNIQUE WITH NOISE REDUCTION,” which is incorporated by reference herein in its entirety for all purposes. In some embodiments, the processor 32 may utilize arbitrary waveform data stored in one or more storage components of the monitor 12, such as the RAM 36, the ROM 38, and/or a mass storage 54 in a feedback loop to modify the modulation of the optical stimulation in near real-time by encoder 28 so as to improve baseline acoustic levels seen by the acoustic detector 18, to optimize the signal-to-noise ratio, signal chain linearity, or to observe a physiologic observable in the received signal at the detector 20 up through digital conversion (e.g., at A/D converter 50), or to compensate for patient-to-patient variation.

In one embodiment, processor 32 may access and execute coded instructions, such as for implementing the algorithms discussed herein, from one or more storage components of the monitor 12, such as the RAM 36, the ROM 38, and/or the mass storage 54. Additionally, the RAM 36, ROM 38, and/or the mass storage 54 may serve as data repositories for information such as templates for LFM reference chirps, coefficient curves, and so forth. For example, code encoding executable algorithms may be stored in the ROM 38 or mass storage device 54 (such as a magnetic or solid state hard drive or memory or an optical disk or memory) and accessed and operated according to processor 32 instructions using stored data. Such algorithms, when executed and provided with data from the sensor 10, may calculate one or more physiological characteristics as discussed herein (such as the type, concentration, and/or amount of an indicator). Once calculated, the physiological characteristics may be displayed on the display 14 for a caregiver to monitor or review. Additionally, the calculated physiological characteristics, such as the hemodynamic parameters, may be sent to a multi-parameter monitor for further processing and display. Alternatively, the processor 32 may use the algorithms to calculate the cardiac output, and the cardiac output may be displayed on the display 14 of the monitor 12.

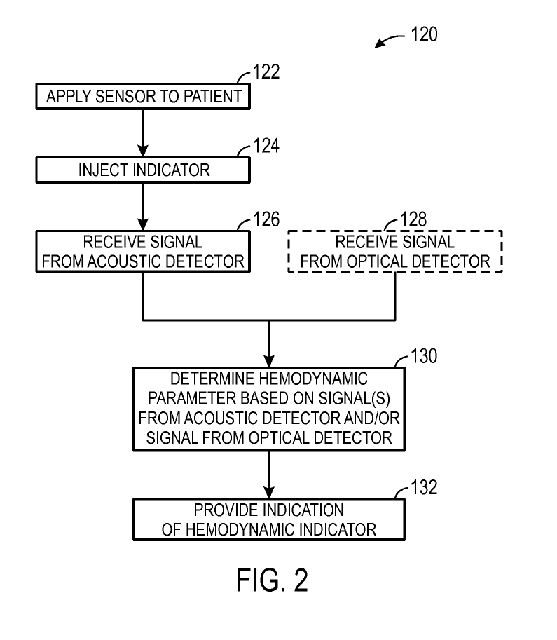

Embodiments of the present disclosure address the problem of noise in the received acoustic signal during photoacoustic measurement of indicator dilution. FIG. 2 is a process flow diagram illustrating a method 120 for determining a physiological parameter (e.g., hemodynamic parameter) from indicator dilution, in conjunction with the photoacoustic sensors 10 and systems 8 as provided. The method is generally indicated by reference number 120 and includes various steps or actions represented by blocks. It should be noted that the method 120 may be performed as an automated procedure by a system, such as system 10. Further, certain steps or portions of the method may be performed by separate devices. For example, a first portion of the method 120 may be performed by a caregiver, while a second portion of the method 120 may be performed by a sensor and/or monitor 12 operating under processor control and in response to signals received from the sensor 10. In addition, insofar as steps of the methods disclosed herein are applied to the received signals, it should be understood that the received signals may be raw signals or processed signals. That is, the methods may be applied to an output of the received signals.

In certain embodiments, the method 120 begins with application of the photoacoustic sensor 10 to the patient 27 at step 122. The sensor 10 emits light into the patient's tissue and detects resulting acoustic waves from the tissue. At step 124, an appropriate indicator is injected or otherwise supplied to the patient 27. In one embodiment, the caregiver may provide an input to the monitor 12 to indicate the indicator injection time point. In certain embodiments, the indicator may be provided as two or more indicators, which may be injected at the same time or different times, according to the desired measured parameter. In one embodiment, the indicator is an isotonic indicator, such as saline. At step 126, a monitoring device, such as the monitor 12, receives a signal from the acoustic detector 20 of the photoacoustic sensor 10 that is representative of detected acoustic waves in the tissue. In certain embodiments, at optional step 128, the monitor may receive a corresponding optical detector signal (i.e., from the same or an overlapping time period as the acoustic detector signal) representative of detected light from the light source 18. Based on the acoustic detector signal and/or the optical detector signal (if present), the desired hemodynamic parameter may be determined at step 130 and an indication of the hemodynamic parameter may be provided by the monitor 12 at step 132.

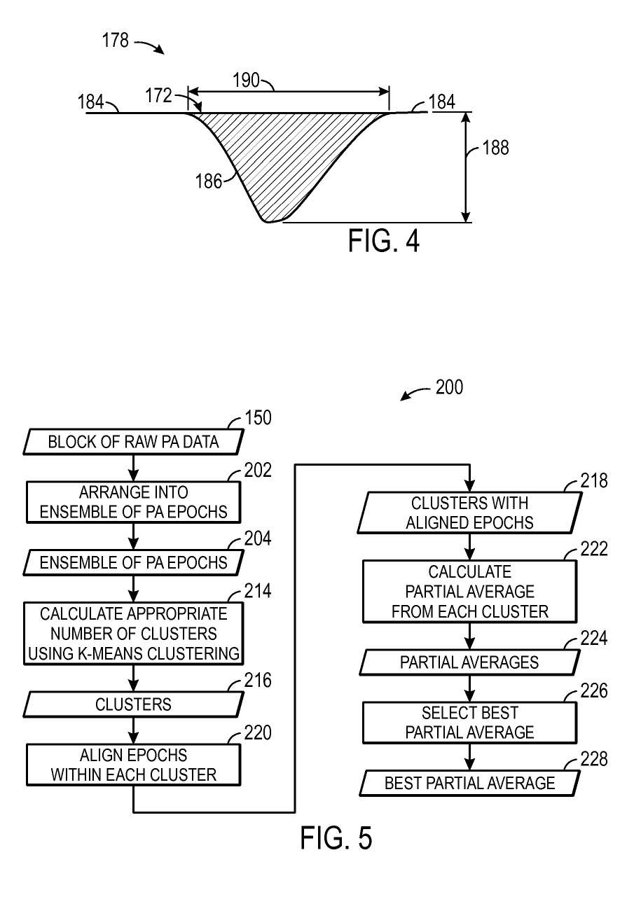

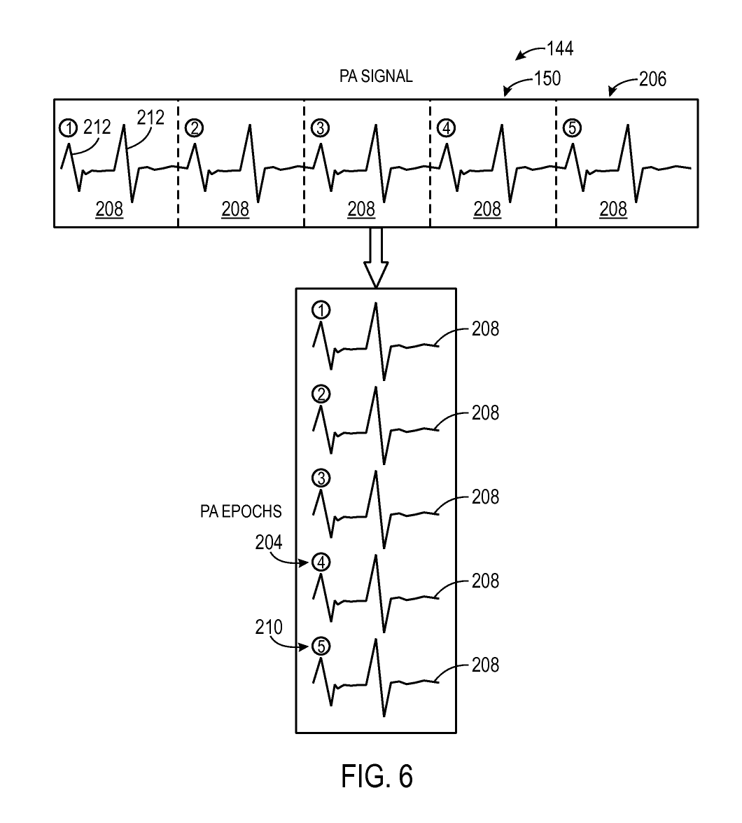

As mentioned above, an ID curve is used to determine cardiac output of a patient. When an indicator is injected into a vein in a cardiovascular system, a diluted temporal profile of the indicator may be measured in a downstream artery. Measurement of this diluted temporal profile may be used to estimate the hemodynamic properties. As described in greater detail below, the ID curve may be calculated from the optical or acoustic signal obtained from the sensor 10. For example, FIG. 6 illustrates an example of a portion of a PA signal of raw PA data 144. The raw PA data 144 may be arranged in a block 150 that includes a number of epochs 208 (labeled 1-5). Some techniques described below, may arrange the epochs 208 into an ensemble 204 (i.e., block 210) prior to calculating the ID curve. As described in greater detail below, one or more features of the ID curve may be utilized to estimate hemodynamic properties. FIG. 4 provides an example of a portion of an ID curve 178 and its features 180. The ID curve 178 includes an identified signal region 172 and a baseline portion 184. Some of the features 180 may include an area 186 of the signal region 172, an amplitude 188 of the signal region 172, and/or a duration 190 of the signal region 172. These features 180 may be used in estimating hemodymanic properties.

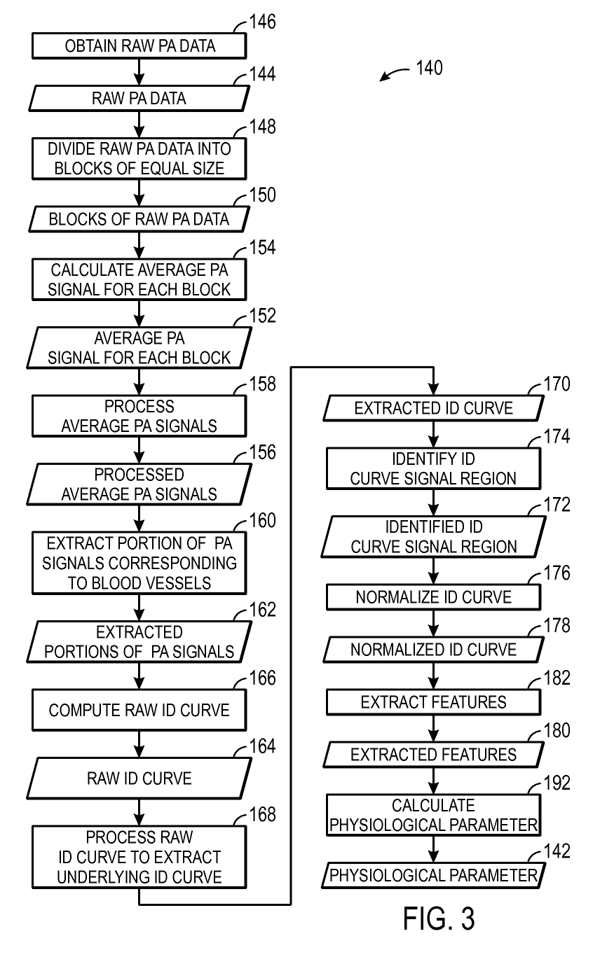

FIG. 3 is a flow diagram of a method 140 for performing step 130 of the method 120 shown in FIG. 2. It should be noted that certain steps or portions of the method 140 may be omitted and/or additional steps may be added. As described in embodiments below, certain techniques utilized for one or more of the steps in the method 140 may reduce the noise in the detected PA signal and/or the resulting ID curve, to enable a more accurate estimation of a physiological parameter 142 such as a hemodynamic parameter (e.g., cardiac output). In certain embodiments, the method 140 includes obtaining raw PA data 144 (e.g., raw acoustic signal data, collected during a single indicator dilution trial or session of a single patient) at step 146. In certain embodiments, the raw PA data 144 may be bandpass-filtered between approximately 0.5 to 5.0 MHz. The raw PA data 144 data may be obtained from the acoustic detector signal from the photoacoustic sensor 10 and is representative of detected photoacoustic waves in the tissue.

At step 148, the raw PA data 144 is divided into blocks 150 of equal size. For example, the blocks 150 may each include a specific number (e.g., same number) of epochs ranging from 2 to 10 or any other number. Each epoch corresponds to a PA response to a single light pulse (see epochs 208 of FIG. 6). The block 150 represents a portion of the acoustic detector signal or PA signal from time point A to time point B and includes the specific number of epochs. An average PA signal 152 for each block 150 may be calculated at step 154. The average PA signal 152 obtained for each block 150 may be processed to obtain processed average PA signals 156 at step 158. At step 160, portions 162 of the processed average PA signals 156 corresponding to the blood vessel may be extracted. Extracting the portions 162 of the PA signals 156 corresponding to the blood vessel removes those portions of the processed average PA signals 156 corresponding to a patient's skin and/or a surface of the photoacoustic sensor 10.

From the extracted portions 162 of the processed average PA signals 156, a raw ID curve 164 may be computed at step 166. At step 168, the raw ID curve 164 may be processed to extract an underlying ID curve 170. Within the extracted underlying ID curve 170, a signal region 172 (i.e., region of curve 170 free from a baseline portion of the curve 170 before the injection and secondary circulation after the injection; see FIG. 4) may be identified at step 174. At step 176, the identified signal region 172 of the ID curve 170 may be normalized to generate a normalized ID curve 178 for the signal region 172. Features 180 may be extracted from the normalized ID curve 178 at step 182.

FIG. 4 illustrates a graphical representation of a portion of the normalized ID curve 178 highlighting features 180 that may extracted from the ID curve 178. The ID curve 178 includes the identified signal region 172 and a baseline portion 184. Some of the extracted features 180 may include an area 186 of the signal region 172, an amplitude 188 of the signal region 172, and/or a duration 190 of the signal region 172. At step 192, the physiological parameter 142 (e.g., hemodynamic parameter such as cardiac output) may be calculated from one or more of the extracted features 180.

Disclosed embodiments of techniques for performing step 154 of the method 140 shown in FIG. 3 may ensure that measures from the PA signal (e.g., average PA signal 152) used in calculating the ID curve 164 are more reliable and robust against jitter due to system noise and other physiological fluctuations. In addition, these techniques may reduce the contamination due to noise sources (e.g., physiological sources and/or measurement errors sources), enhance the SNR of the ID curve 164, and improve the estimation accuracy of the physiological parameter 142 (e.g., cardiac output). For example, FIG. 5 illustrates a flow diagram of method 200 that utilizes k-means clustering approach to calculate the average PA signal 152 for each block 150 of raw PA data 144. The steps of the method 200 may be repeated for each block 150 of raw PA data 144 to calculate the average PA signal 152. In certain embodiments, the raw PA data 144 used in method 200 may be bandpass-filtered between approximately 0.5 to 5.0 MHz. In one embodiment, the method 200 includes arranging raw PA data epochs within the block 150 into an ensemble 204 at step 202. FIG. 6 illustrates the block 150 of raw PA data 144 prior to the ensemble arrangement (i.e., block 206) and after arranging the epochs 208 (labeled 1-5) into the ensemble 204 (i.e., block 210). As illustrated in FIG. 6, each epoch 208 includes a number of peaks 212 that represent the acoustic response to the optical signal by the blood vessel, skin, and/or surface of the photoacoustic sensor 10. The number of peaks 212 in each epoch 208 may range from two to three.

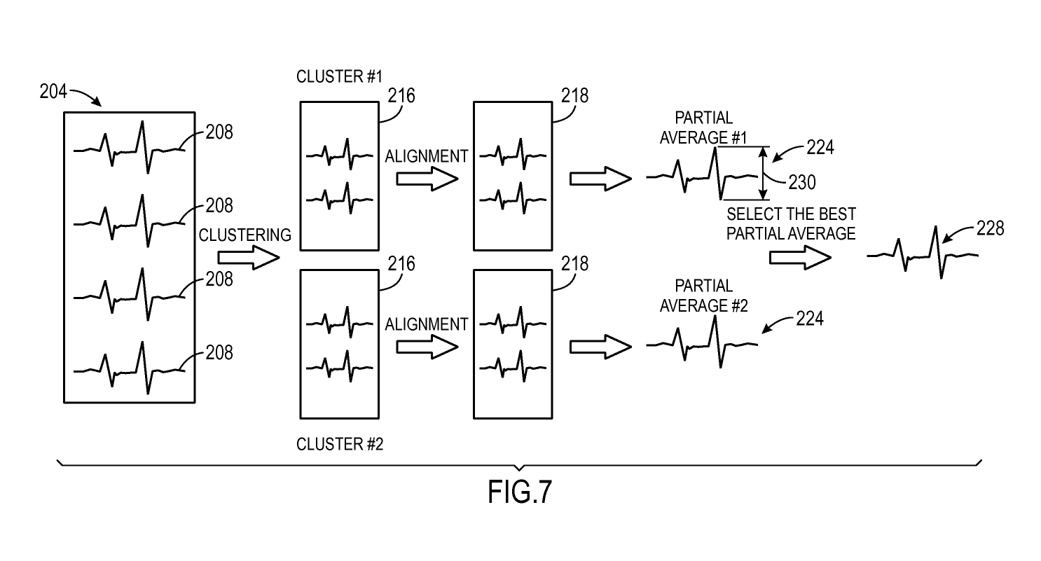

Returning to FIG. 5, at step 214, an appropriate number of clusters 216 may be calculated from the ensemble 204 of PA epochs 208 using a k-means clustering-based approach. As illustrated in FIG. 7, the PA epochs 208 of the ensemble 204 are arranged into the appropriate number of clusters 216. The epochs 208 within each of the clusters 216 may be aligned to generate clusters 218 with aligned epochs 208 at step 220 of FIG. 5 (see FIG. 7). Woody filtering or a correlation-based approach may be utilized to align the epochs 208 for performing step 220. At step 222, a partial average PA signal 224 may be calculated for each cluster 218 by averaging all of the aligned epochs 208 within a respective cluster 218 (see FIG. 7). From the partial average PA signals 224 from the clusters 218, a best partial average PA signal 228 may be selected for a respective block 150 of raw PA data 144 to represent the average PA signal 152 at step 226 (see FIG. 7). In certain embodiments, for each partial average PA signal 224 from the clusters 218, a peak-to-peak amplitude 230 (see FIG. 7) or other measure may be calculated from the region of the signal 224 corresponding to the blood vessel. The partial average PA signal 224 with the largest peak-to-peak amplitude 230 may be selected as the best partial average signal 228.

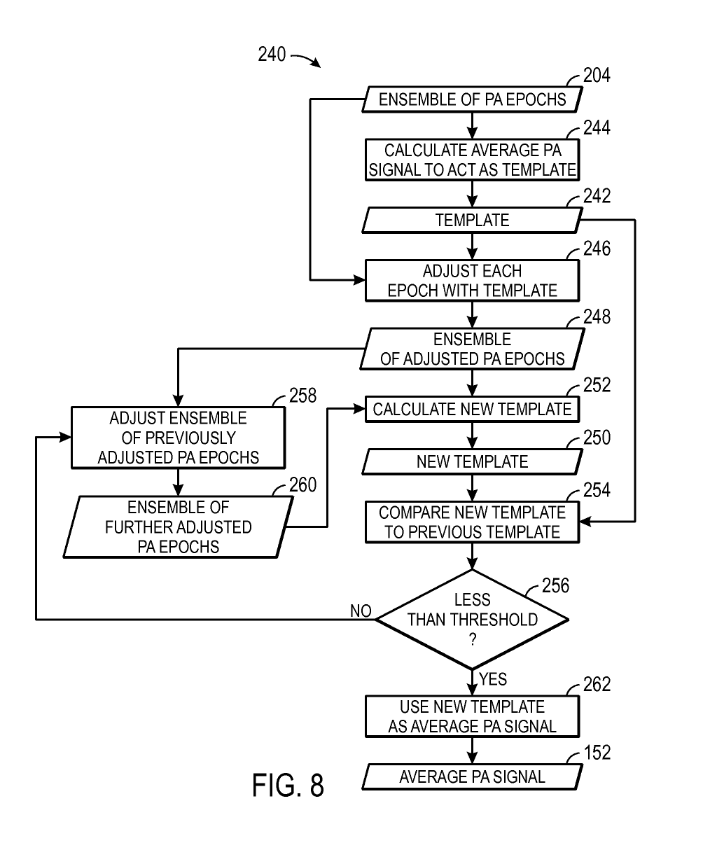

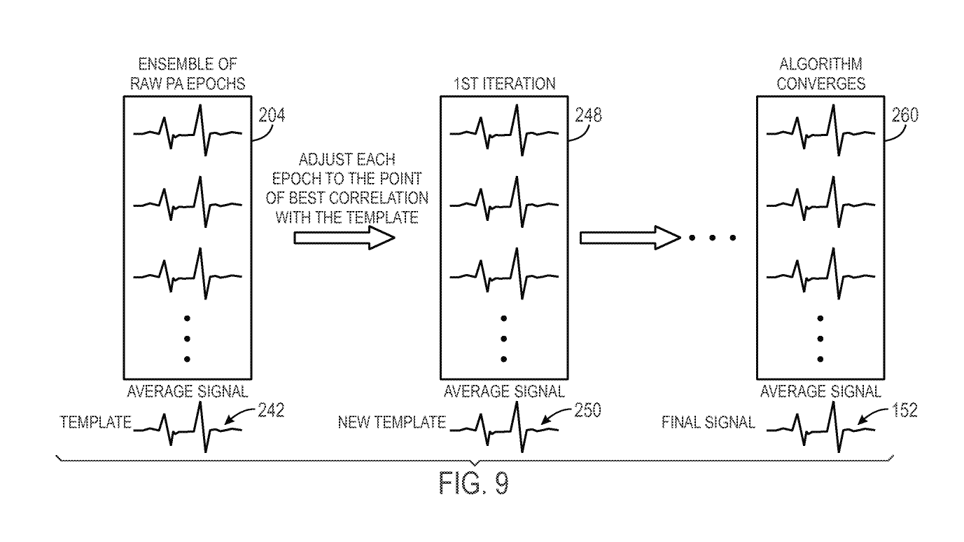

In one embodiment, instead of k-means clustering, a Woody-filtering based approach may be utilized to calculate the average PA signal 152 for each block 150 of raw PA data 144. For example, FIG. 8 illustrates a flow diagram of a method 240 that utilizes Woody filtering in calculating the average PA signal 152. The steps of the method 240 may be repeated for each block 150 of raw PA data 144 to calculate the average PA signal 152. In certain embodiments, the raw PA data 144 used in method 240 may be bandpass-filtered between approximately 0.5 to 5.0 MHz. In one embodiment, the method 240 includes calculating an initial average PA signal to act as a template 242 from the ensemble 204 of PA epochs 208 at step 244. The ensemble 204 of PA epochs 208 may be obtained as described in step 202 of the method 200. Utilizing the template 242, each epoch 208 of the ensemble 204 may be adjusted at step 246. Adjustment of an individual epoch 208 may include calculating a correlation between the epoch 208 and the template 242 for all lags (e.g., correlation lags) in both directions and aligning the epoch 208 by the lag that corresponds to the point of highest or maximum correlation with the template 242. This adjustment may be performed on each epoch 208 of the ensemble 204 to generate an ensemble 248 of adjusted epochs 208 as illustrated in FIG. 9. A new template 250 (i.e., new average PA signal) may be calculated from the ensemble 248 of adjusted epochs 208 at step 252 (see FIG. 9 graphically representing the calculation of the average PA signal 152 utilizing Woody filtering). At step 254, the new template 250 may be compared the previous template 242 (or template 250 for subsequent iterations). Comparison of the new template 250 and the previous template 242 may include determining the mean square value between the templates 242, 250 and determining if the mean square value is less than a threshold (e.g., predetermined threshold) at step 256. If mean square value is not less than the threshold, the ensemble 248 of previously adjusted epochs 208 may be adjusted with the new template 250 to generate another ensemble 260 of further adjusted epochs 208 at step 258 and steps 254 and 256 repeated. If the mean square value is less than the threshold (demonstrating convergence of the algorithm), the template 250 may be used as the average PA signal 152 at step 262 (see FIG. 9).

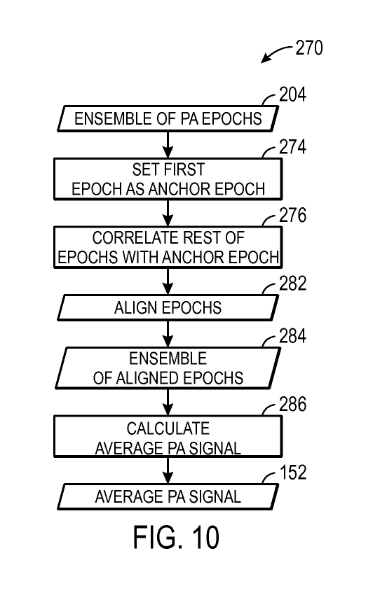

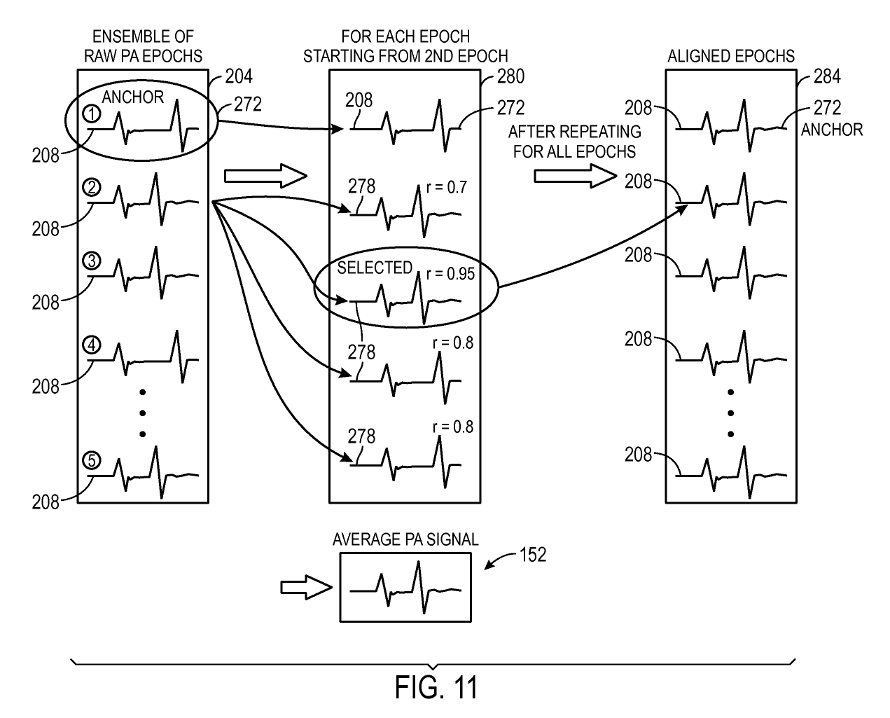

In one embodiment, instead of Woody filtering or k-means clustering, a correlation-based approach may be utilized to calculate the average PA signal 152 for each block 150 of raw PA data 144. For example, FIG. 10 illustrates a flow diagram of a method 270 that utilizes the correlation-based approach in calculating the average PA signal 152. The steps of the method 270 may be repeated for each block 150 of raw PA data 144 to calculate the average PA signal 152. In certain embodiments, the raw PA data 144 used in method 270 may be bandpass-filtered between approximately 0.5 to 5.0 MHz. In one embodiment, the method 270 includes setting the first epoch 208 (i.e., first temporally within the block 150 of epochs 208 and labeled 1 in the ensemble 204) as the anchor epoch 272 at step 274 (see FIG. 11 graphically representing the calculation of the average PA signal 152 utilizing the correlation-based approach). The ensemble 204 of PA epochs 208 may be obtained as described in step 202 of the method 200. The rest of the epochs 208 (e.g., correlating epochs 208), starting with the second epoch 208 (i.e., second temporally and labeled 2), may be separately correlated with the anchor epoch 272 at step 276. In certain embodiments, the epoch 208 (e.g., second epoch 208, third epoch 208, etc.) is correlated to the anchor epoch 272 for lags 278 (e.g., correlation lags) in both directions. For example, the correlation lags 278 may be generated by moving the correlating epoch 208 in time by a number of lags in both directions. A correlation coefficient (e.g., ranging between −1 to +1) may be determined for different correlation lags 278 of a respective correlating epoch 208. For example, FIG. 11 illustrates an ensemble 280 of the anchor epoch 272, the second epoch 208, and the corresponding correlation lags 278 of the second epoch 208. At step 282, the correlation lag 278 with the highest correlation score may be selected for each respective correlating epoch 208 and aligned with respect to the anchor epoch 278 to generate an ensemble 284 of aligned epochs 208 (see FIG. 11). For example, as shown in FIG. 11, the correlation lag 278 with the highest correlation (e.g., highest correlation coefficient) for the second epoch 208 is utilized as the epoch 208 within ensemble 284 for alignment. The average PA signal 152 may be calculated from the ensemble 284 of aligned epochs 208 at step 286 (see FIG. 11).

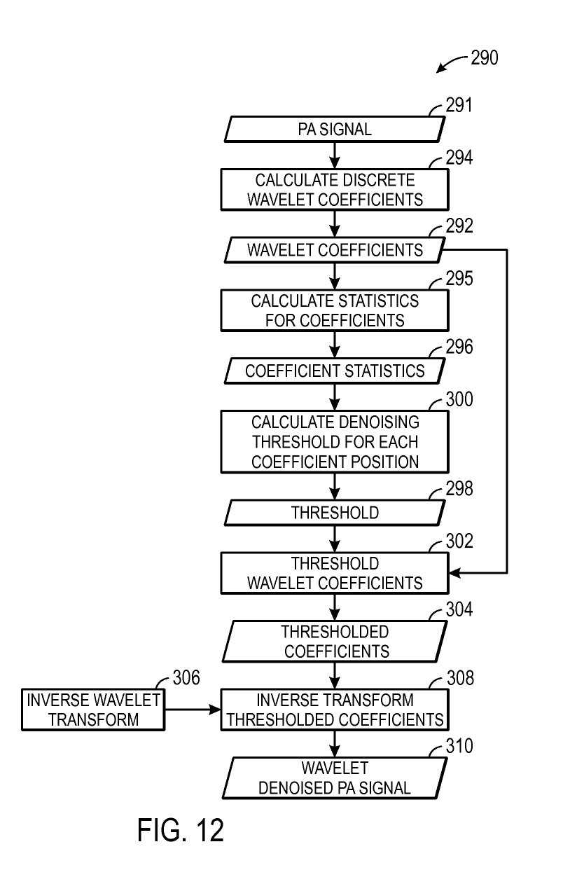

The following disclosed embodiments of a technique for performing step 158 of the method 140 shown in FIG. 3 may reduce the contamination due to noise sources (e.g., physiological sources and/or measurement errors sources), enhance the SNR of the ID curve 166, and/or improve the estimation accuracy of the physiological parameter 142 (e.g., cardiac output). For example, FIG. 12 illustrates a flow diagram of a method 290 that utilizes a wavelet-based approach to denoise a PA signal 291 prior to calculating the raw ID curve 164. The PA signal 291 utilized in the method 290 may be the average PA signals 152 for the blocks 150. The average PA signals 152 may be obtained as described in the methods 200, 240, and 270. In certain embodiments, the PA signal 291 utilized in the method 290 may be the ensemble 204 of PA epochs 208. The ensemble 204 of PA epochs 208 may be obtained as described in step 202 of the method 200. In one embodiment, the method 290 includes calculating discrete wavelet coefficients 292 from the PA signal 291 (e.g., ensemble 204 of PA epochs 208 or average PA signal 152) at step 294. At step 295, coefficient statistics 296 may be calculated for each epoch 208 or average PA signal 152 using an appropriate wavelet (e.g., biorthogonal B-spline wavelet). The coefficient statistics 296 may include a mean, μᵢ, and/or standard deviation, σᵢ, at coefficient position i across the epochs 208 or average PA signals 152. Based on the coefficient statistics 296, a threshold 298 (e.g., denoising threshold) is calculated for each coefficient position i at step 300, where the threshold 298 is μᵢ/σᵢ. At step 302, the wavelet coefficients 292 are thresholded to generate thresholded coefficients 304. Thresholding the wavelet coefficients 292 may include comparing each coefficient 292 at position i to its respective threshold 298. If the coefficient 292 is below the threshold 298, the coefficient 292 may be set to zero. A step 308, an inverse wavelet transform 306 may be applied to the thresholded coefficients 304 to inverse transform the coefficients 304 to generate a wavelet denoised PA signal 310 (e.g., wavelet denoised epochs or wavelet denoised average PA signals) from which the raw ID curve 164 may be calculated.

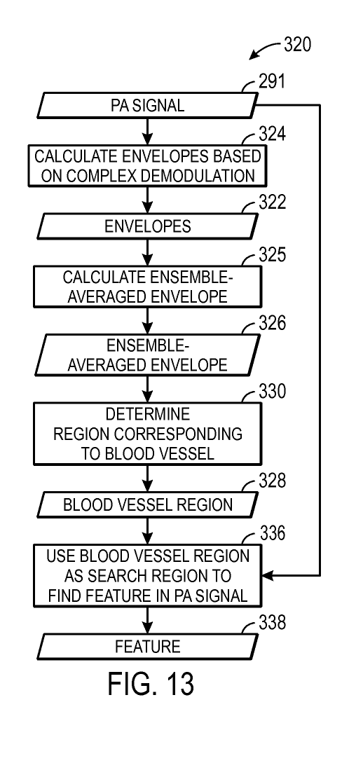

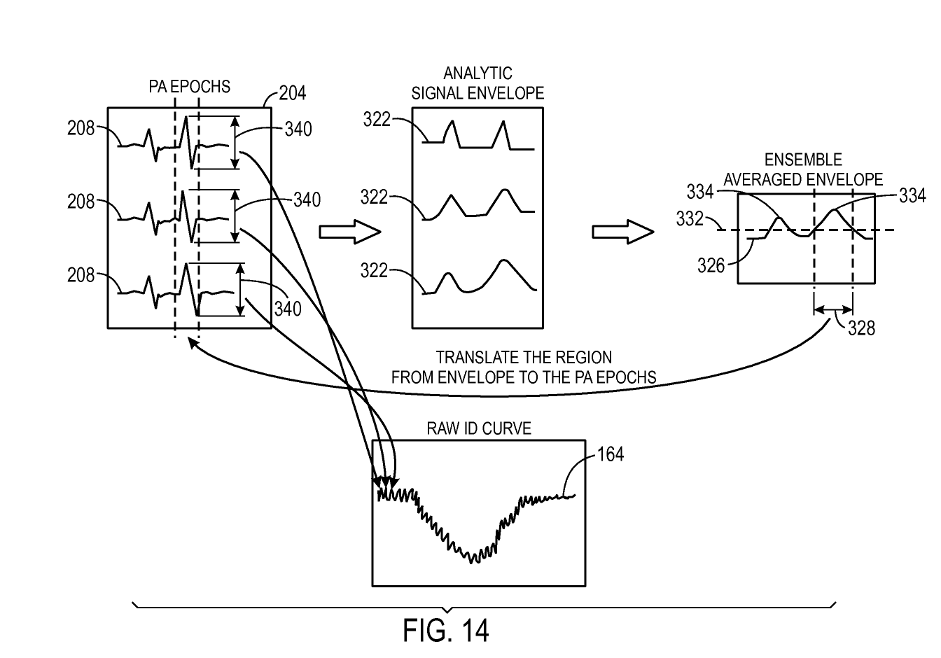

The following disclosed embodiments of a technique for performing step 160 of the method 140 shown in FIG. 3 may reduce the contamination due to noise sources (e.g., skin, sensor surface, etc.) not related to the blood response in the PA signal used in calculating the ID curve 164. This may enhance the SNR of the ID curve 166, and improve the estimation accuracy of the physiological parameter 142 (e.g., cardiac output). For example, FIG. 13 illustrates a flow diagram of a method 320 that utilizes a complex demodulation-based approach to segment the PA signal 291 for a region corresponding to a blood vessel prior to calculating the raw ID curve 164. In certain embodiments, the PA signal 291 may be the wavelet denoised PA signal 310 obtained in the method 290. In some embodiments, the PA signal 291 utilized in the method 320 may be the average PA signals 152 for the blocks 150. The average PA signals 152 may be obtained as described in the methods 200, 240, and 270. In certain embodiments, the PA signal 291 utilized in the method 320 may be the ensemble 204 of PA epochs 208. The ensemble 204 of PA epochs 208 may be obtained as described in step 202 of the method 200. In one embodiment, the method 320 includes calculating envelopes 322 (e.g., analytic signal envelopes) based on complex demodulation for the PA signal 291 (e.g., for each epoch 208 or average PA signal 152) at step 324. Envelope calculation may include computing the analytic signal (e.g., complex signal consisting of an input epoch 208 or average PA signal 152 in its real part and the Hilbert transform of the epoch 208 or average PA signal 152 in its imaginary part) using complex demodulation followed by finding the magnitude of the analytic signal to generate the envelope 322. At step 325, an ensemble-averaged envelope 326 may be calculated from the envelopes 322. FIG. 14 illustrates an example of utilizing the complex demodulation-based approach to generate an ensemble-averaged envelope 326 from the ensemble 204 of PA epochs 208 as described in the method 320. A region 328 corresponding to a blood vessel may be determined at step 330. Determining the blood vessel region 328 may include comparing the ensemble-averaged envelope 326 to a preset threshold 332 (e.g., a percentage of a maximum magnitude such as 5%) to find all of the regions 334 of the envelope 326 that exceed the threshold 332 (see FIG. 14). From those regions 334, the region 334 closest to an expected time of occurrence of blood vessel response is selected as the blood vessel region 328 (see FIG. 14). At step 336, the blood vessel region 328 from the ensemble-averaged envelope 326 may be used as a search region to find features 338 (e.g., global peak-to-peak 340 as shown in FIG. 14, area, etc.) of the PA signal 291 (e.g., each epoch 208 or average PA signal 152) to be used in calculating the raw ID curve 164. For example, the feature 338 of each epoch 284 or average PA signal 152 may be translated into a single sample or point for the raw ID curve 164.

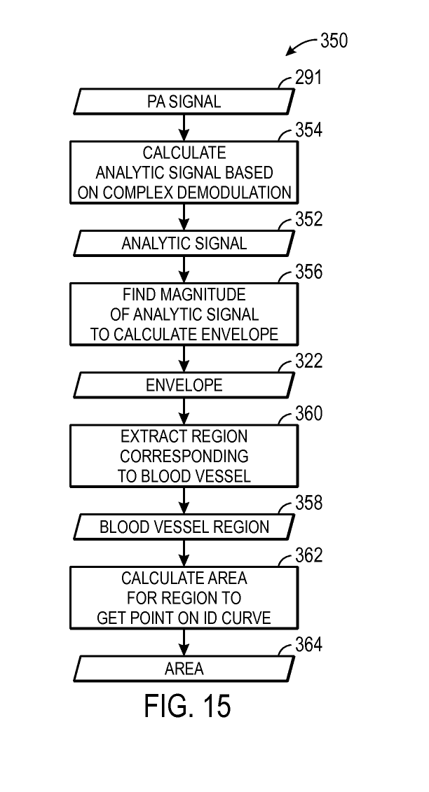

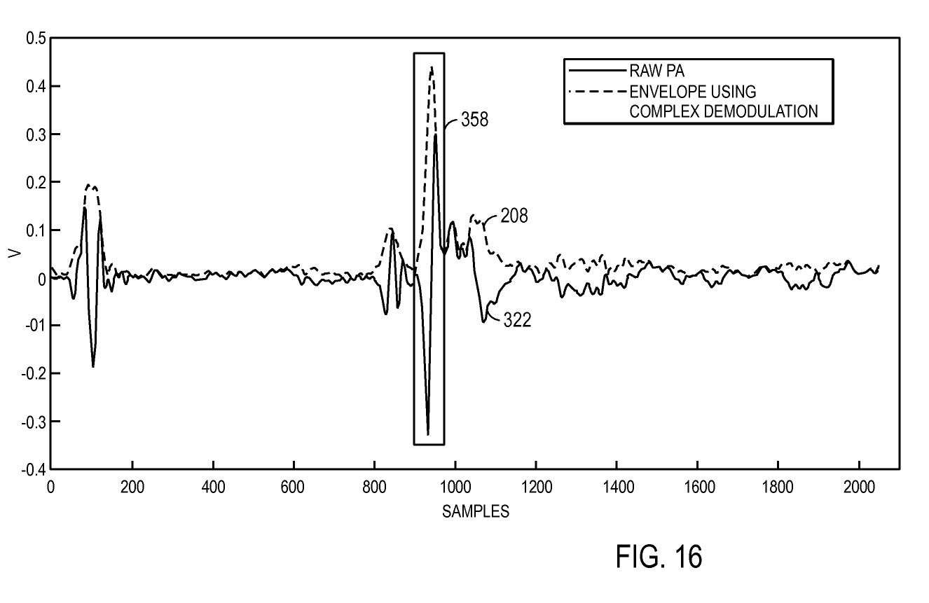

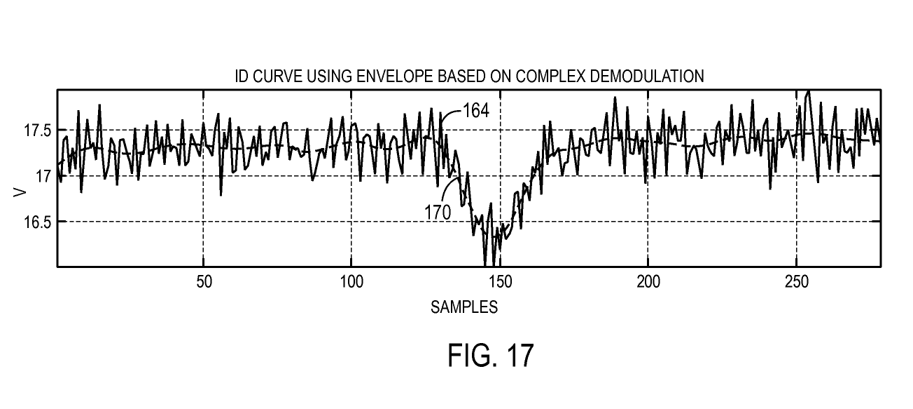

Besides global peak-to-peak, an area of the envelope 322 may be used in calculating the raw ID curve 164 from the PA signal 291. The area of the envelope 322 may enable the capture of morphological aspects of the PA signal 291 not taken into account with global peak-to-peak values. FIG. 15 illustrates a flow diagram of a method 350 for utilizing a complex demodulation-based approach in calculating the raw ID curve 164. In one embodiment, the method 350 includes calculating the envelope 322 for the PA signal 291 (e.g., epoch 208 or average PA signal 152 or their wavelet denoised equivalents) as described in step 325 of the method 320. For example, an analytic signal 352 may be calculated from the PA signal 291 (e.g., for each epoch 208 or average PA signal 152) based on complex demodulation at step 354. The analytic signal 352 is a complex signal consisting of an input epoch 208 or average PA signal 152 in its real part and the Hilbert transform of the epoch 208 or average PA signal 152 in its imaginary part. At step 356, finding a magnitude of the analytic signal 352 may be used to generate the envelope 322. A region 358 of the envelope 322 corresponding to the blood vessel may be extracted at step 360. For example, the region 358 may be extracted using a technique similar to step 330 in the method 320. At step 362, an area 364 (i.e., sum of the envelope values within a specific time window) for the blood vessel region 358 of the envelope 322 may be calculated to get a point (i.e., sample) on the raw ID curve 164. FIG. 16 illustrates an example of the raw PA epoch 208 (represented by the solid line), the envelope 322 (represented by the dashed line) for the epoch 208, and the region 358 (represented by the rectangle) of the envelope 322 used for calculating the area 364. FIG. 17 illustrates an example of the raw ID curve 164 (represented by the solid line) generated using the method 350 and a filtered or underlying ID curve 170 (represented by the dashed line) of the raw ID curve 164.

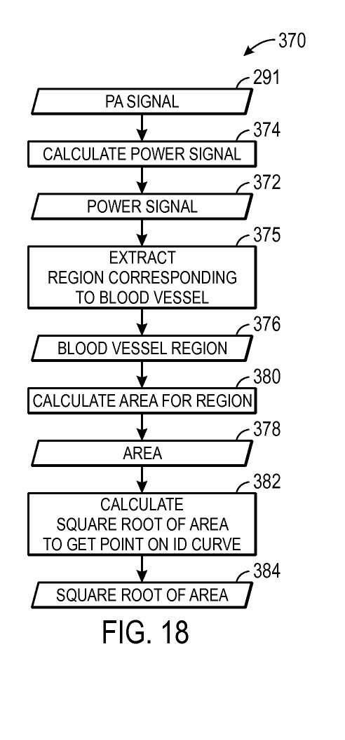

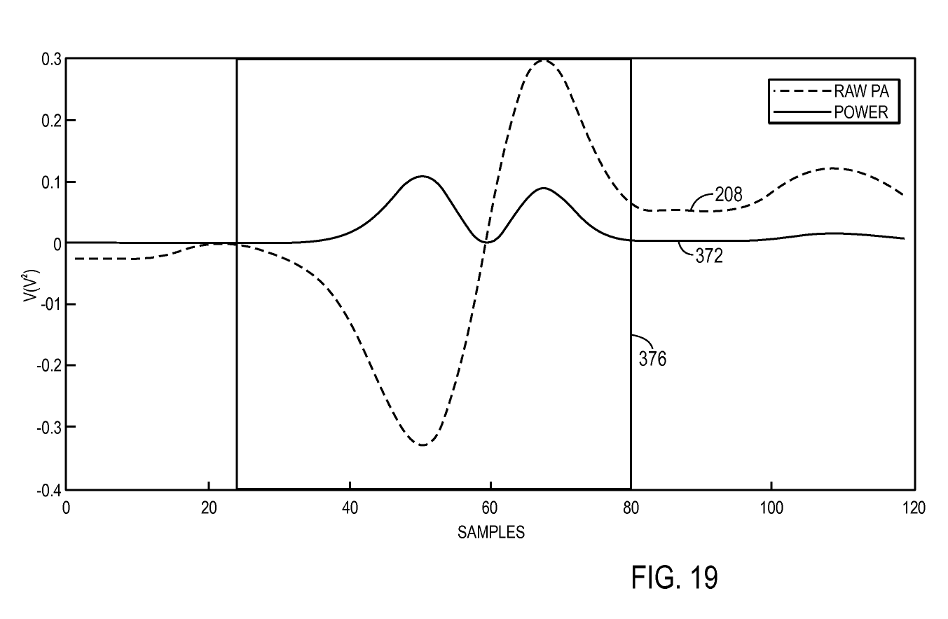

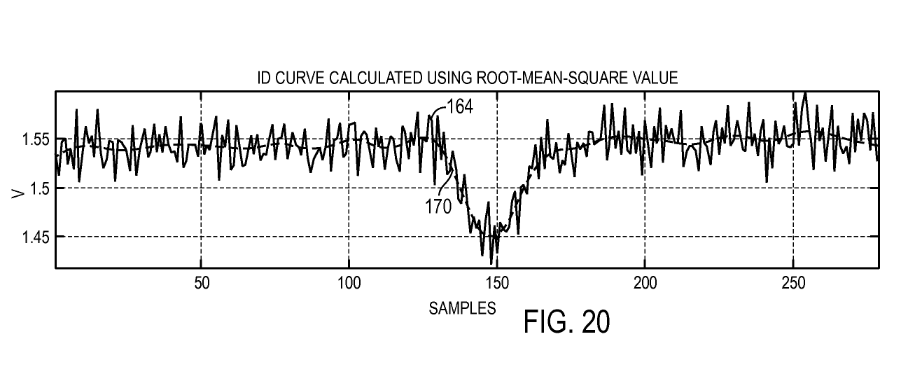

In one embodiment, an area of a power envelope may be used in calculating the raw ID curve 164 from the PA signal 291. The area of the power envelope may enable the capture of morphological aspects of the PA signal 291 not taken into account with global peak-to-peak values. FIG. 18 illustrates a flow diagram of a method 370 for utilizing a power signal 372 (i.e., power envelope) in calculating the raw ID curve 164. In one embodiment, the method 370 includes calculating the power signal 372 for the PA signal 291 (e.g., epoch 208 or average PA signal 152 or their wavelet denoised equivalents) at step 374. For example, the power signal 372 may be calculated by squaring the PA signal 291. In step 375, a region 376 of the power signal 372 corresponding to the blood vessel may be extracted. For example, the region 376 may be extracted using a technique similar to step 330 in the method 320. An area 364 (i.e., sum of the power signal values within a specific time window) for the blood vessel region 376 of the power signal 372 may be calculated at step 380. At step 382, a square root 384 of the area 364 may be calculated to get a point (i.e., sample) on the raw ID curve 164. FIG. 19 illustrates an example of the raw PA epoch 208 (represented by the dashed line), the power signal 372 (represented by the solid line) for the epoch 208, and the region 376 (represented by the rectangle) of the power signal 372 used for calculating the area 378. FIG. 20 illustrates an example of the raw ID curve 164 (represented by the solid line) generated using the method 370 and a filtered or underlying ID curve 170 (represented by the dashed line) of the raw ID curve 164.

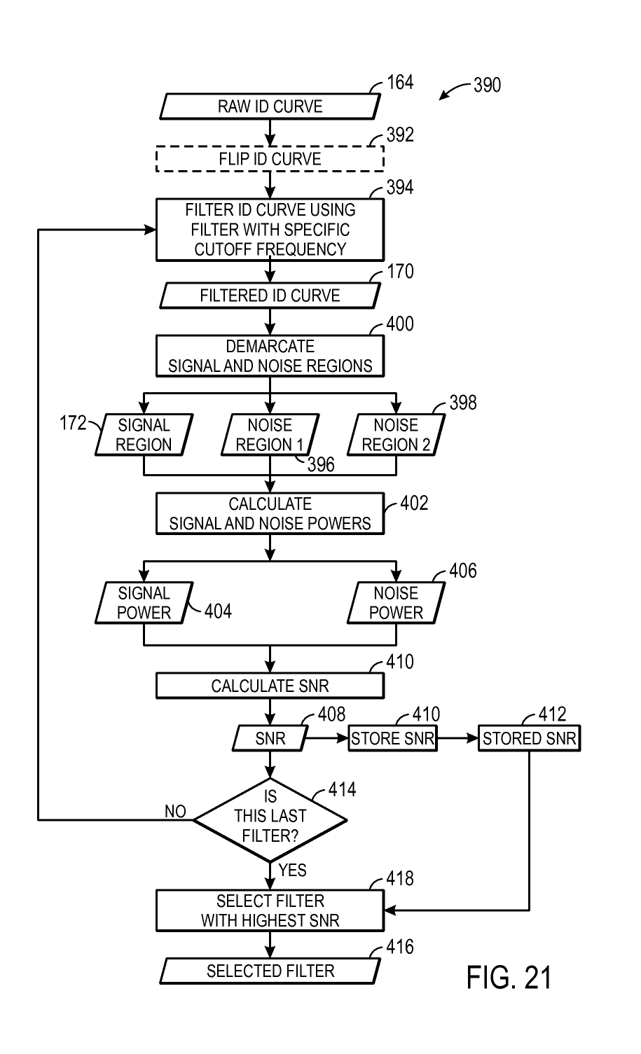

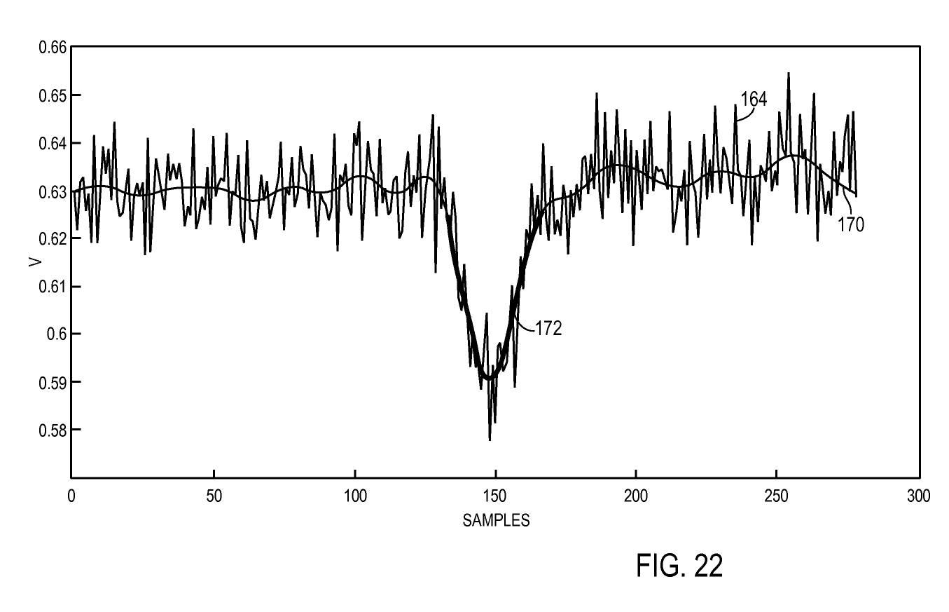

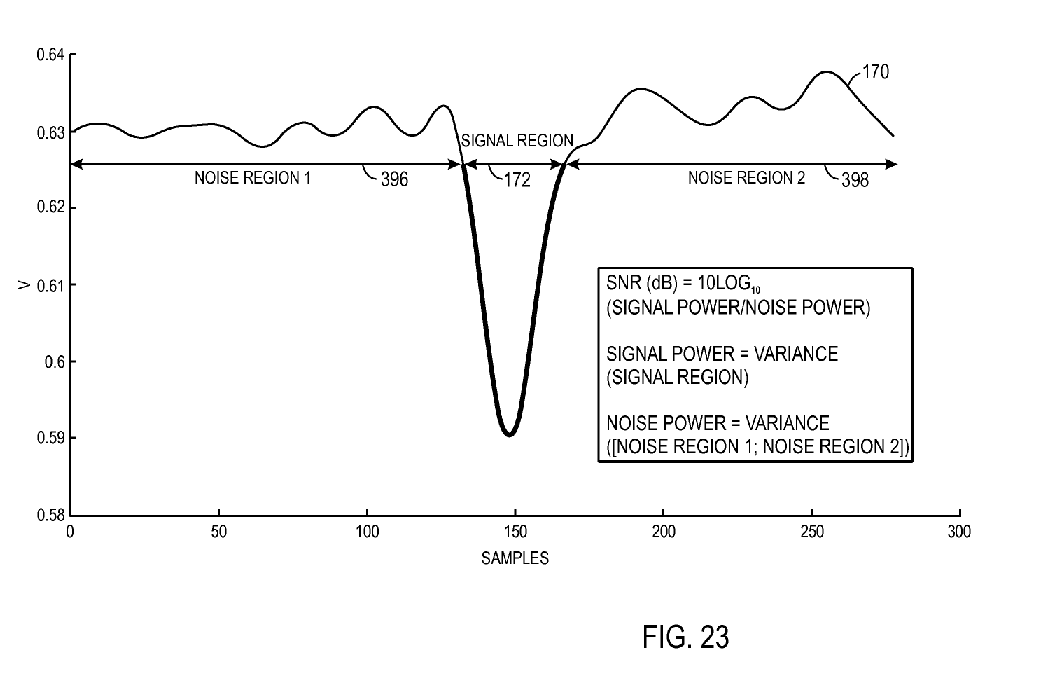

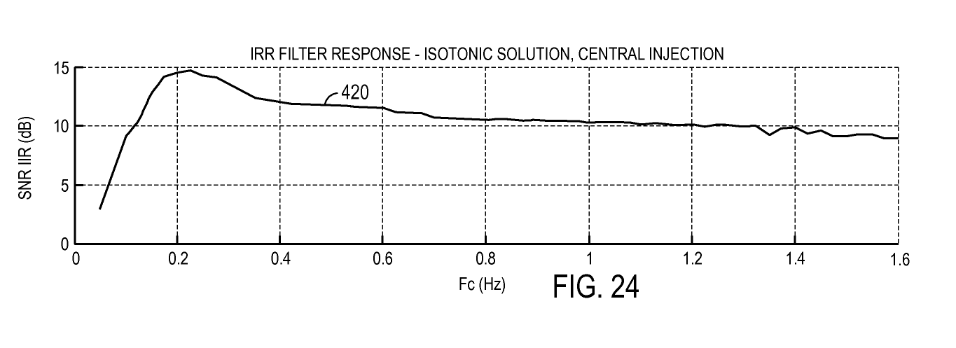

The following disclosed embodiments of a technique for performing step 168 of the method 140 shown in FIG. 3 to denoise the ID curve 164 by demarcating the signal region of the ID curve 164 from a baseline before the injection of the indicator and second circulation after the injection. In addition, the technique may reduce the contamination due to noise sources (e.g., physiological sources such as respiratory artifact and heart activity), enhance the SNR of the ID curve 164, and improve the estimation accuracy of the physiological parameter 142 (e.g., cardiac output). In certain embodiments, the raw ID curve 164 may be derived from PPG data obtained from the optical signal from the photoacoustic sensor 10 representative of detected light from the light source 18. For example, FIG. 21 illustrates a flow diagram of a method 390 that utilizes adaptive filter selection to denoise the raw ID curve 164. In one embodiment, the method 390 utilizes the raw ID curve 164 derived from raw PA data 144 as described in the techniques above. In another embodiment, the raw ID curve 164 may be derived from raw PPG data obtained from the optical signal. A PPG derived ID curve may be approximately 180 degrees out of phase with respect to a PA derived ID curve. If the raw ID curve 164 is derived from raw PPG data, the ID curve 164 may be flipped at step 392. At step 394, the ID curve 164 is filtered using a filter with a specific cutoff frequency to generate the filtered (e.g., extracted or underlying) ID curve 170. FIG. 22 illustrates an example of the raw ID curve 164 and the filtered ID curve 170 from a single specific filter at a particular cutoff frequency, where the signal region 172 of the filtered ID curve 170 is represented by the thicker solid line. The filtered ID curve 170 may be demarcated into the signal region 172 and noise regions 396, 398 (i.e., noise regions 1 and 2, respectively) at step 400 of the method 390 as illustrated in FIG. 23. Noise regions 396, 398 correspond to the baseline prior to the injection of the indicator and secondary circulation after injection of the indicator, respectively. At step 402, the signal power 404 and the noise power 406 may be calculated from the signal region 172 and the noise regions 396, 398, respectively. For example, the signal power 404 may be calculated as the variance of the signal region 172. The noise power 406 may be calculated as the variance of both noise regions 396, 398. From the signal power 404 and the noise power 406, a SNR 404 for the filter with the specific cutoff frequency may be calculated at step 410 using the following:

$$S N R ( dB ) = 10 \times {log}_{10} ( \frac{{P}_{signal}}{{P}_{noise}} ) , \qquad ( 1 )$$

where P_{signal} represents the signal power 404 and Pₙₒᵢₛₑ represents the noise power 406. It should be noted that the signal region 172 is not totally free from baseline noise and the SNR 404 calculated is an estimate of the true SNR. At step 410, the SNR 404 for the filter with the specific cutoff frequency may be stored with other SNRs 412 (e.g., stored SNRs) for other filters with different cutoff frequencies. Upon determining the SNR 410 for the filter with the specific cutoff frequency, the method 390 may including determining whether the filter with the specific cutoff frequency was the last filter of the filters with the different cutoff frequencies that the SNR 410 still needs to be calculated for at step 414. If there are remaining filters for determining the SNR 404, the above steps of method 390 may repeated for each of the remaining filters with different cutoff frequencies. If there are no remaining filters for determining the SNR 404, the filter (i.e., selected filter 416) with the highest SNR may be selected from among the stored SNRs 412 for the different filters with the different cutoff frequencies at step 418 and used in the subsequent processing of the ID curve 164. FIG. 24 illustrates a curve 420 representing the SNRs 410 for filters with different cutoff frequencies. This curve 420 may aid in selecting the optimal filter from among the filters with the different cutoff frequencies. Adaptive filter selection enables the utilization of a quality metric to select the filter that improves denoising of the ID curve 164 to generate the underlying curve 170.

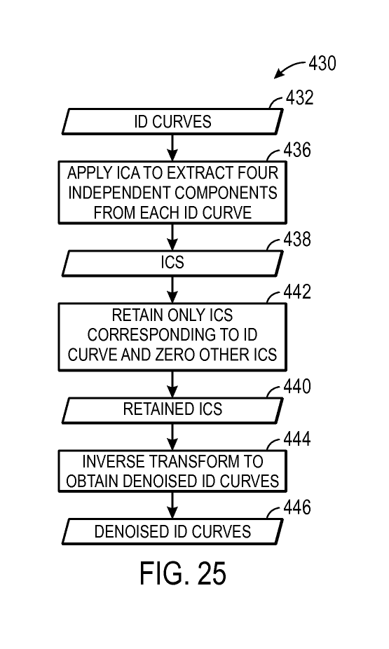

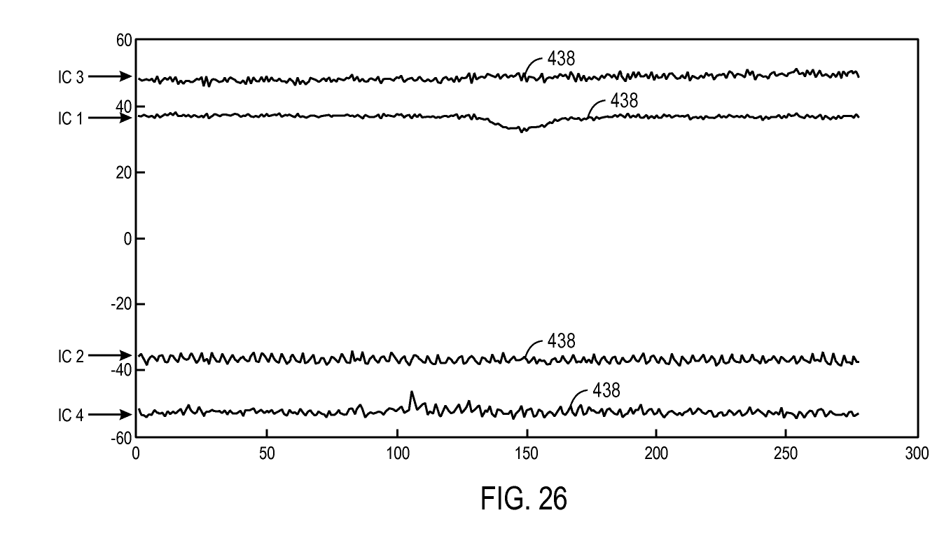

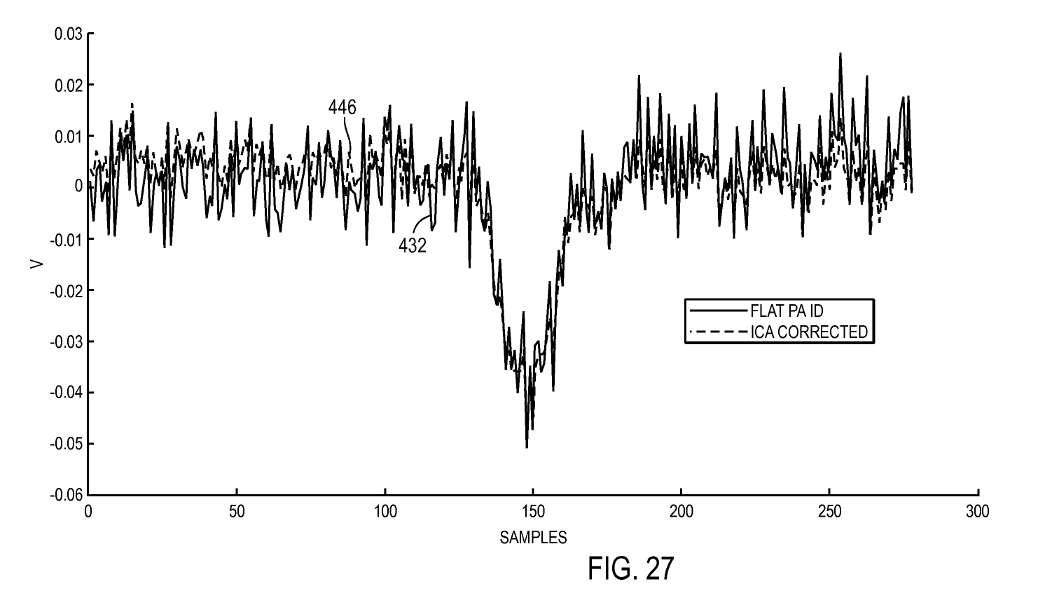

FIG. 25 illustrates a flow diagram of a method 430 of utilizing an independent component analysis (ICA)-based approach for denoising ID curves 432. Utilizing a blind source separation technique such as ICA enables spatial filtering via sampling of signals and noise across multiple channels to get a better estimate of both. At step 436, ICA may be applied to the ID curve 432 to generate four independent components (ICs) 438 from the ID curve 432. FIG. 26 illustrates the ICs 438 (e.g., IC 1, IC 2, IC 3, IC 4) extracted from a single ID curve 432. IC1 corresponds to the underlying ID signal, while IC 2, IC 3, and IC 4 correspond to noise sources. Only the IC(s) 438 (i.e., retained ICs 440) from the ID curve 432 corresponding to the ID curve 432 (e.g., IC 1 in FIG. 26) may be retained, while the rest of the ICs 438 (e.g., IC 2, IC 3, and IC 4 in FIG. 26) are eliminated or zeroed at step 442. At step 444, the one or more retained ICs 440 corresponding to the ID curve 432 may be inverse transformed (i.e., projected back to the signal domain) to obtain denoised ID curves 446. FIG. 27 illustrates the ID curve 432 prior to denoising, represented by a solid line, and the denoised curve 446 (i.e., ICA corrected as described in the method 430), represented by a dashed line.

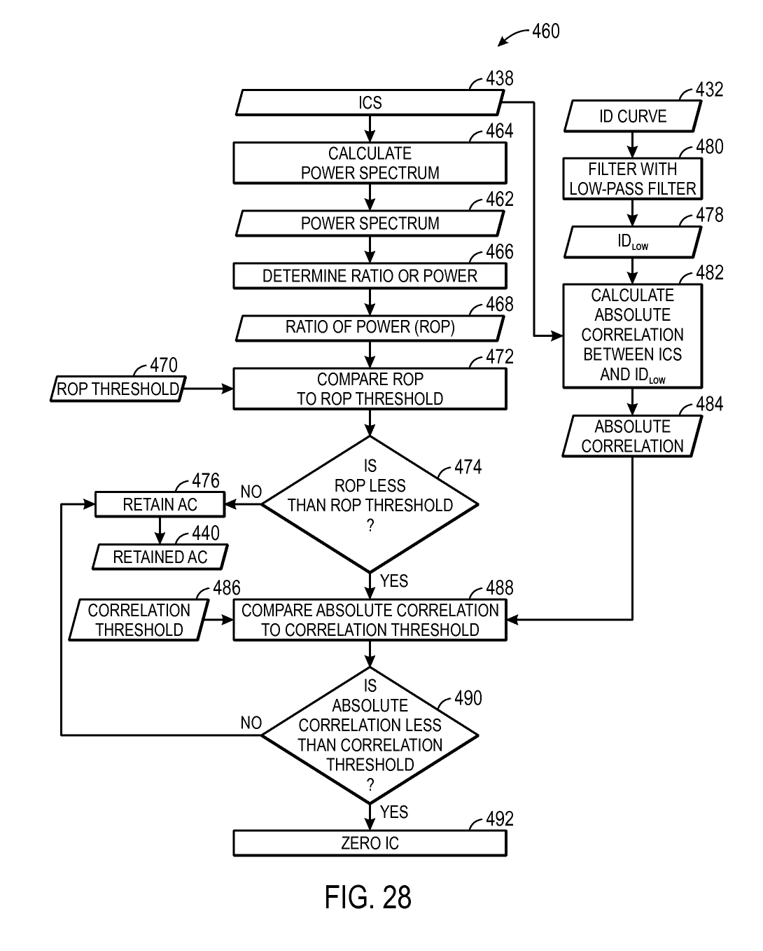

FIG. 28 is a flow diagram of a method 460 for performing step 442 of the method 430 shown in FIG. 25. In one embodiment, the method 460 includes calculating a power spectrum 462 (e.g., via a Fourier transform) for each of the ICs 438 (e.g., IC 1, IC 2, IC 3, IC 4 in FIG. 26) for a particular ID curve 432 at step 464. At step 466, a ratio of power (ROP) or noise power ratio 468 may be determined for each IC 438 for frequencies between, e.g., 0.2 Hz and the maximum power, from its respective power spectrum 462. The ROP 468 for each IC 438 may be compared to a predetermined threshold (e.g., ROP threshold) 470 a step 472. At step 474, the comparison may include determining whether the ROP for a respective IC 438 is less than the threshold 470. Any ICs 438 (e.g., retained ICs 440) with the ROP 468 less than the threshold 470 may be retained at step 476 for generating the denoised ID curve 446 as described in method 430. If the ROP 468 for a particular IC 438 is not less than the threshold 470, additional factors may be analyzed (e.g., such as those immediately below) in determining whether to retain the IC 438.

In one embodiment, the method 460 also includes low-pass filtering the ID curve 432 to obtain a low-pass filtered ID curve (ID_{low}) 478 at step 480. At step 482, an absolute correlation 484 may be calculated between the ICs 438 and the low-pass filtered ID curve 478. The absolute correlation 484 may be the absolute value of a correlation coefficient (e.g., ranging between 0 and 1) calculated between the IC 438 and the low-pass filtered ID curve 478. If the ROP 468 for a particular IC 438 is not less than the threshold 470, the absolute correlation 484 between the particular IC 438 and the low-pass filtered ID curve 478 may be compared to a predetermined threshold (e.g., correlation threshold) 486 at step 488. At step 490, the comparison may include determining whether the absolute correlation 484 between a respective IC 438 and the low-pass filtered ID curve 478 is less than the threshold 486. Any ICs 438 (e.g., retained ICs 440) with the absolute correlation 484 less than the threshold 486 may be retained at step 476 for generating the denoised ID curve 446 as described in the method 430. If the absolute correlation 484 between a particular IC 438 and the low-pass filtered ID curve 478 is not less than the threshold 470, the particular IC 438 may be zeroed or eliminated at step 492. In certain embodiments, steps 488 and 490 may be performed prior to or simultaneous with steps 472 and 474.

Besides adaptive filter selection and ICA, a total variation minimization-based approach may be utilized for performing step 168 of the method 140 shown in FIG. 3 to reduce the contamination due to noise sources (e.g., physiological sources such as respiratory artifact and heart activity), enhance the SNR of the ID curve 164, and improve the estimation accuracy of the physiological parameter 142 (e.g., cardiac output). Total variation is the amount that a function (e.g., signal such as the ID curve 164) goes up and down. In particular, total variation in a single dimension may be described as

$$\int \vert \frac{d f}{d t} \vert d t . \qquad ( 2 )$$

The total variation adds up all changes in the function, positive or negative, across the entire domain. Since it does not matter how the value of the function changes, but only the total change, the sharp edges in the signal (e.g., ID curve 164) are not penalized. In particular, the total variation will remove the extra oscillations without penalizing the underlying sharp signal features. The total variation minimization-based approach enables denoising of the raw ID curve 164 (e.g., derived from raw PA or raw PPG data) and extracting of the underlying ID signal or curve 170. The total variation minimization problem is formulated as a convex optimization problem as in

minimize Σᵢ∥gᵢ₊₁−gᵢ∥₂ (3)

subject to the following mean squared error constraint

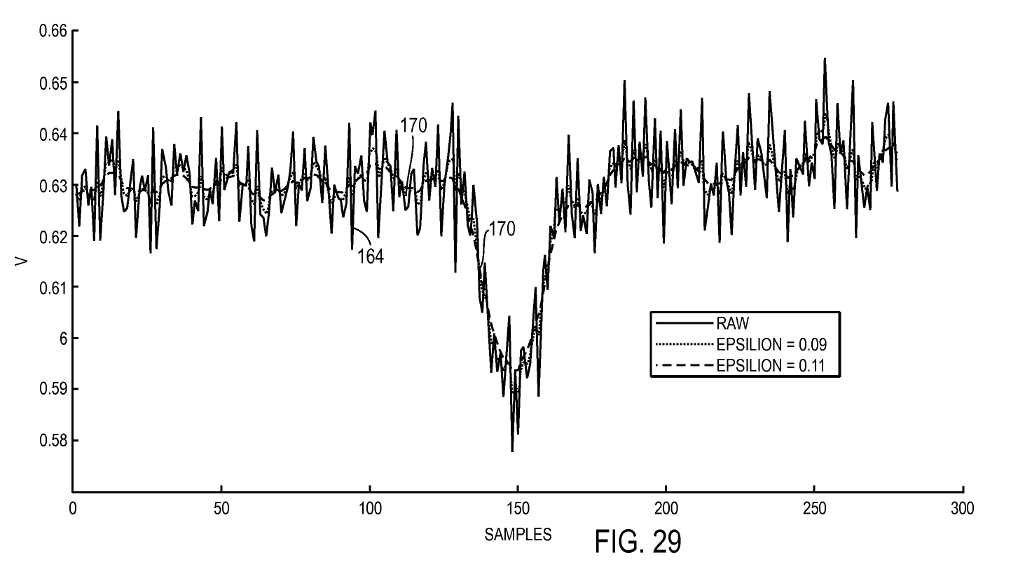

∥g−x∥₂≦ε, (4)

where x represents the raw ID curve 164, g represents the underlying ID curve 170 filtered using total variation minimization, and ε represents the error threshold. FIG. 29 illustrates an example of the raw ID curve 164 (represented by a solid line) and a couple of filtered or denoised curves 170 for ε=0.09 (represented by a dotted line) and ε=0.11 (represented by a dashed line). The total variation minimization process for ε=0.09 results in less overall smoothing, while ε=0.11 results in more overall smoothing. In both instances, the total variation minimization process removes noise oscillations without compromising the underlying ID curve 170. In one embodiment, an adaptive filter selection approach similar to method 390 may be utilized to select (e.g., adaptively select) the optimum value for ε in the above total variation minimization process that will give the best SNR for the underlying ID curve 170. Instead of utilizing different filters with different cutoff frequencies, different values for ε may be used. The ε with the highest SNR may be utilized in denoising the raw ID curve 164.

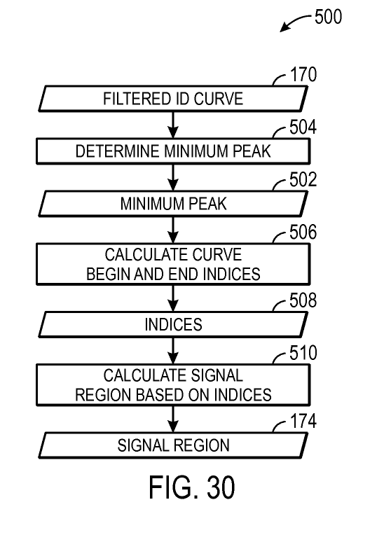

The following disclosed embodiment of a technique for performing step 174 of the method 140 shown in FIG. 3 may demarcate the signal region 174 of the filtered (e.g., low-pass filtered) or underlying ID curve 170 from a baseline before the injection of the indicator and second circulation after the injection without introducing artifacts (e.g., introduced by fit-based techniques). In addition, the technique may reduce the contamination due to noise sources (e.g., physiological sources such as respiratory artifact and heart activity), enhance the SNR of the ID curve 164, and improve the estimation accuracy of the physiological parameter 142 (e.g., cardiac output). For example, FIG. 30 illustrates a flow diagram of a method 500 that utilizes a minimum peak 502 of the ID curve 170 for calculating the signal region 174. In one embodiment, the method 390 utilizes the ID curve 170 derived from PA data. In another embodiment, the ID curve 170 is derived from PPG data. PPG derived ID curves may be flipped as described above prior to performing the method 500. In one embodiment, the method 500 includes determining the minimum peak 502 of the ID curve 170 at step 504. At step 506, indices 508 (e.g., curve begin and curve end indices) may be calculated based on the minimum peak 502. For example, the indices 508 may include the relative value (e.g., percentage) of each sample on the ID curve 170 relative to the minimum peak 502. From the indices 508, the signal region 174 of the ID curve 170 may be calculated at step 510. For example, the beginning of the single region 174 may be defined as the last sample of the ID curve 170 in the curve begin index that is greater than or equal 10% of a minimum value before the minimum peak 502. The end of the signal region 174 may be defined as the first sample greater than or equal to 10% of a minimum value after the minimum peak 502. FIG. 22 illustrates an example of the raw ID curve 164 and the filtered ID curve 170, where the signal region 172 of the filtered ID curve 170 is represented by the thicker solid line.

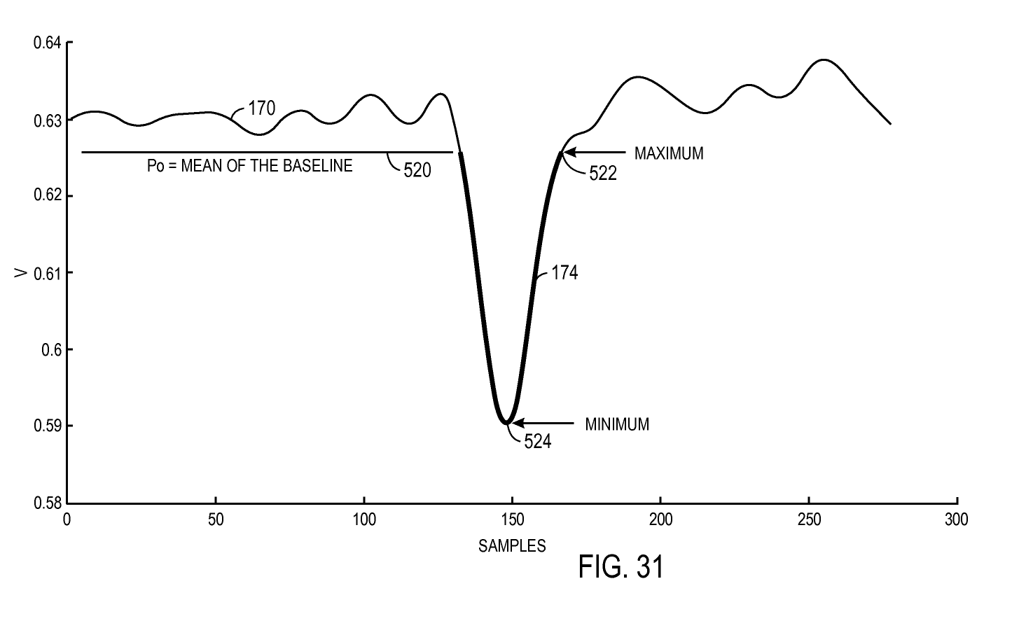

The following disclosed embodiments of techniques for performing step 176 of the method 140 shown in FIG. 3 may normalize the signal region 174, represented by Pa in FIG. 31, based solely of features of the signal region 174. Typically, the signal region 174 of the ID curve 170 is normalized relative to a mean 520 of a baseline prior to the single region 174, represented by Po in FIG. 31, as

$${Pa}_{normalized} = 1 - \frac{Pa}{Po} . \qquad ( 5 )$$

However, solely utilizing the features of the signal region 174 for normalization may improve the estimation accuracy of the physiological parameter 142 (e.g., cardiac output) and eliminate the dependence on a stable baseline. In addition, features outside of the signal region may be more susceptible to noise and instability. In one embodiment, a maximum 522 (see FIG. 31) may be determined for the signal region 174, and then the signal region 174 is normalized with respect to the maximum 522 as in

$${Pa}_{normalized} = 1 - \frac{Pa}{max ( Pa )} . \qquad ( 6 )$$

In another embodiment, a minimum 524 (see FIG. 31) may be determined for the signal region 174, and then the signal region 174 is normalized with respect to the minimum 524 as in

$${Pa}_{normalized} = 1 - \frac{Pa}{min ( Pa )} . \qquad ( 7 )$$

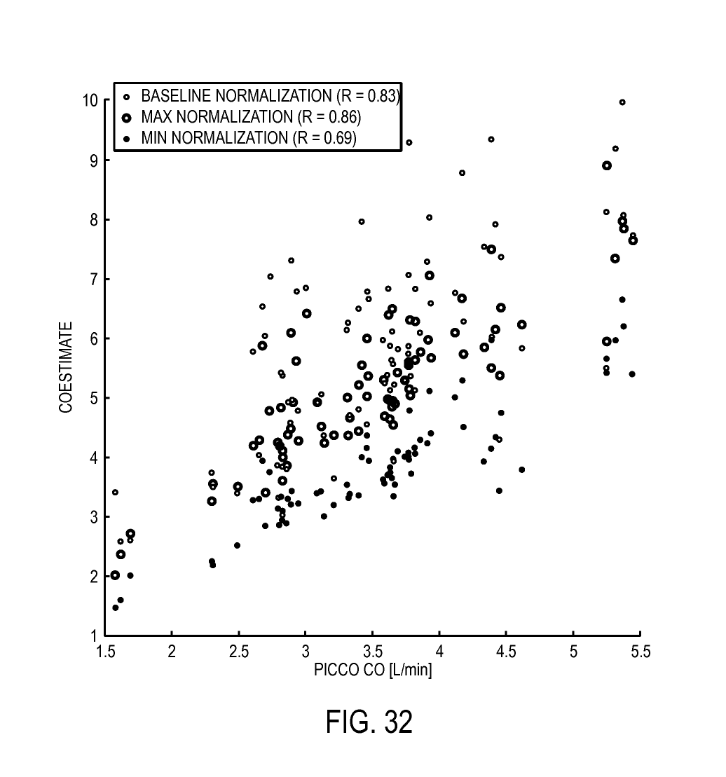

FIG. 32 illustrates how the different embodiments of normalization (i.e., solely utilizing the features of the signal region 174) compare relative to one another. Specifically, FIG. 32 compares cardiac output estimates derived from signal regions 174 of ID curves 170 normalized as described above with respect to the cardiac output measured using a separate device (e.g., PICCO™ device).

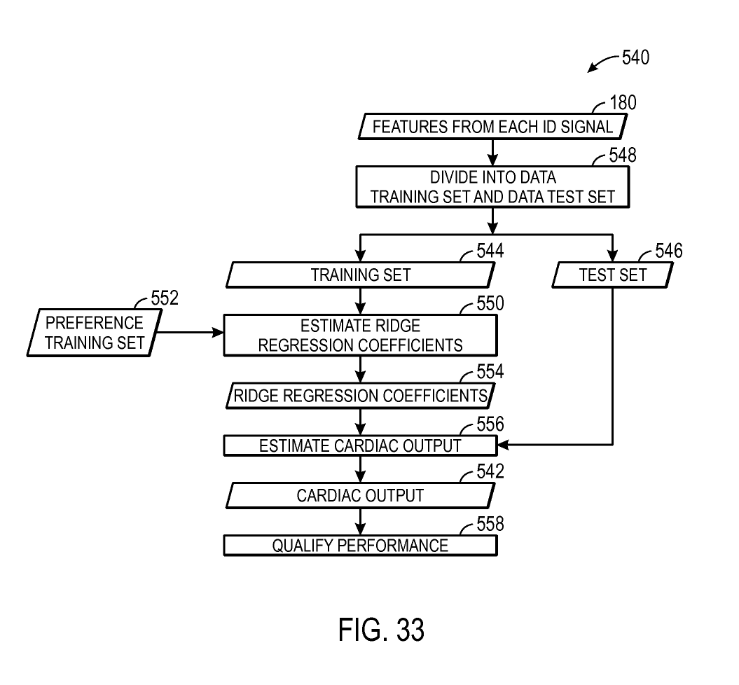

The following disclosed embodiment of a technique for performing step 192 of the method 140 shown in FIG. 3 may utilize a machine-learning approach (as opposed to a model-based approach) to correct for bias (e.g., present in model-based results) and to improve the estimation accuracy of the physiological parameter 142 (e.g., cardiac output). FIG. 33 is a flow diagram of a method 540 of utilizing ridge regression (i.e., machine-learning approach) to estimate cardiac output 542. In one embodiment, one or more features 180 of the signal region 174 for each ID curve 170 are divided into a data training set 544 and a data test set 546 for cross-validation at step 548. As illustrated in FIG. 4, some of the extracted features 180 may include the area 186 of the signal region 172, the amplitude 188 of the signal region 172, and/or the duration 190 of the signal region 172. At step 550, in conjunction with a reference data training set 552 (e.g., cardiac output data from a separate device such as a PICCO™ device), ridge regression coefficients 554 may be estimated from the data training set 544. Ridge regression coefficients 554 may be determined using the following optimization problem

minimize∥yₜᵣₐᵢₙ−Xₜᵣₐᵢₙw∥²+λ∥w²∥, (8)

where vector yₜᵣₐᵢₙ represents the reference data training set 552, matrix Xₜᵣₐᵢₙ represents the training set features (i.e., training set 544), scalar λ represents the regularization factor, and vector w represents the coefficient (i.e., ridge regression) vector. The solution to the above optimization problem may be as follows:

w*=(Xₜᵣₐᵢₙ^{T}Xₜᵣₐᵢₙ+λI)⁻¹Xₜᵣₐᵢₙ^{T}yₜᵣₐᵢₙ, (9)

where T represents the transpose operator and w* represents the estimate of w. At step 556, the cardiac output 542 for the data test set 546 may be estimated as

yₜₑₛₜ=Xₜₑₛₜw*, (10)

where matrix Xₜₑₓₜ represents the test set features (i.e., test set 546) and yₜₑₛₜ represents the estimated cardiac output 542. Multiple rounds (e.g., 10 rounds) of cross-validation may be performed using different divisions of the features 180 of the ID curves 170 into the training set 544 and the test set 546. In the different rounds of cross-validation, a different single subject and/or different single sample may be removed from the data prior to division into the training set 544 and the test set 546. The cross-validation results (i.e., cardiac output estimates 542) may be averaged over the rounds.

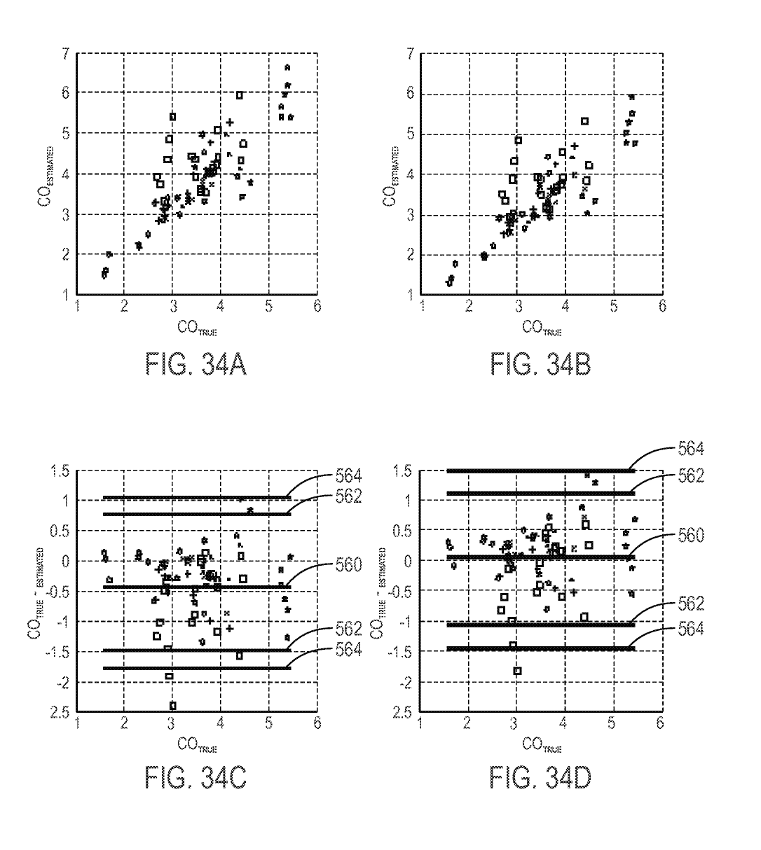

At step 558, the performance of the ridge regression-based approach may be quantified. For example, cardiac output estimates 542 obtained may be evaluated against actual cardiac output measurements (e.g., from a PICCO™ device) via obtaining a Pearson correlation coefficient and/or determining a percentage of samples (i.e., cardiac output estimates 542) within ±1.42 liters per minute. Comparisons of cardiac output estimates obtained in a conventional equation-based or model-based approach against estimates 542 obtained using ridge regression are illustrated in FIGS. 34A-D. FIG. 34A and FIG. 34B illustrate, respectively, scatter plots for both the conventional equation-based approach (shown in FIG. 34A) and the ridge-regression based approach (shown in FIG. 34B) against the actual cardiac output measurements. FIG. 34C and FIG. 34D illustrate, respectively, Bland-Altman plots for both the conventional equation-based approach (shown in FIG. 34C) and the ridge-regression based approach (shown in FIG. 34D) against the actual cardiac output measurements. In the Bland-Altman plots, line 560 represents the bias, lines 562 represent the bias±2σ, and lines 564 represent bias±1.42 liters per minute. The plots in FIGS. 34A-D indicate that ridge regression corrects for bias present in model-based results.

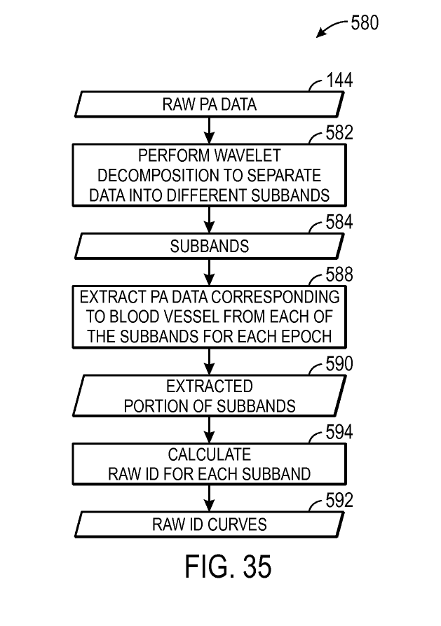

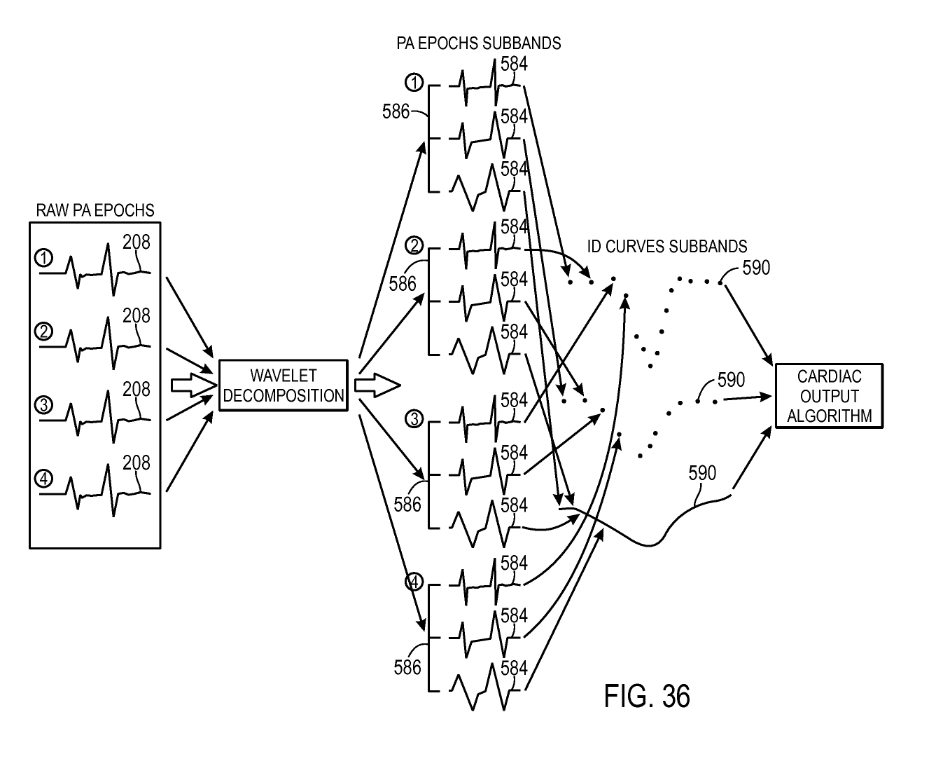

FIG. 35 is a flow diagram of another method 580 for performing step 130 of the method 120 shown in FIG. 2 utilizing a wavelet-based subband approach. It should be noted that certain steps or portions of the method 580 may be omitted and/or additional steps may be added. Utilizing the wavelet-based approach described below, may provide access to subtle changes in ID curve morphology across various frequency bands and, thus, provide overall more information to generate the physiological parameter 142 such as a hemodynamic parameter (e.g., cardiac output). In one embodiment, wavelet decomposition is performed on raw PA data or PA signal 144 at step 582. In certain embodiments, as described above, the raw PA data 144 may divided into blocks 150 including a specific number of epochs 208. Wavelet decomposition may result in the generation of multiple subbands 584 for different frequencies for each epoch 208 as illustrated in FIG. 36. In FIG. 36, each epoch 208 (labeled 1-4) includes a respective group 586 (labeled 1-4) of the subbands 584 at different frequencies generated via wavelet decomposition. At step 588, portions 590 of each of the subbands 584 for each epoch 208 corresponding to the blood vessel may be extracted. Extracting the portions 590 corresponding to the blood vessel removes those portions of the subbands 584 corresponding to a patient's skin and/or a surface of the photoacoustic sensor 10. Extraction of the portions 590 may be performed utilizing the techniques described above. From the extracted portions 590 of the subbands 584, a raw ID curve 592 may be calculated at step 594. Specifically, a different ID curve 592 may be calculated for each subband frequency utilizing subbands 584 derived from multiple epochs 208. For example, as illustrated in FIG. 36, the top subband 584 from each group 586, the middle subband 584 from each group 586, and the bottom subband 584 from each group 586 may respectively be utilized to generate the top, middle, and bottom ID curves 592. The top, middle, and bottom subbands 584 in FIG. 36 may each represent a different frequency within a group 586, while the frequency of the corresponding top, middle, and bottom subbands 584 of the groups 586 may be the same. The ID curves 592 corresponding to each subband frequency may be utilized as described in method 140 and the other techniques described above to generate the physiological parameter 142 such as a hemodynamic parameter (e.g., cardiac output).

As discussed herein, the disclosed noise reduction techniques may be used to calculate physiological parameters, such as hemodynamic parameters. Accordingly, the disclosed embodiments may use the corrected and/or denoised acoustic detector signal as an input to hemodynamic parameter algorithms where the PA detector signal or the PA signal is denoted as an input. For example, the denoised PA detector signal may be used to determine cardiac output when the PA source is fully affected by the indicator dilution (e.g., a majority of the arterials vessels are sensitive to the injected indicator) as disclosed in U.S. patent application Ser. No. 13/836,531, entitled “PHOTOACOUSTIC MONITORING TECHNIQUE WITH NOISE REDUCTION,” which is incorporated by reference herein in their entirety for all purposes. However, in certain situations, the arterial vessels may be partially sensitive to the injected indicator and generate a relatively lower PA signal (e.g., relative to when a majority of arterial vessels are sensitive to the injected indicator. For example, a blood vessels wall and/or a portion of blood adjacent the blood vessel wall (due to laminar blood flow) may be insensitive to the injected indicator.

The following technique accounts for when the PA source is partially sensitive to the injected indicator to calculate physiological parameters, such as hemodynamic parameters (e.g., cardiac output). In one embodiment, if V_{I}, the amount of an isotonic solution, is instantaneously injected at t=0 (i.e. the time of starting the injection is set to zero), the blood flow rate at the measurement point for the PA signal is:

$$F = \frac{{V}_{It}}{{\int }_{0}^{∞} Δ \frac{{V}_{I} ( t )}{Δ V} d t} \qquad ( 11 )$$

where ΔV and ΔV_{I}(t) are blood volume and isotonic volume rates during the unit time interval, Δt, respectively, passing through the exit sectional area of the arterial vessel. Equation (11) indicates that the whole isotonic saline indicator passes through the outlet as time goes to infinity, which assumes that no isotonic molecules interact with tissues outside the cardiovascular system. Note that ΔV_{I}(t)/ΔV in the denominator in Eq. (11) is the isotonic solution concentration c(t). A PA signal is proportional to an absorption coefficient, μₐ of artery blood that is also proportional to a total hemoglobin concentration, c_{tHb} in the unit blood volume ΔV in the vessel. Therefore, the background PA signal before the indicator injection can be

$${PA}_{b} = K \frac{{tHb}_{b}}{V} + {PA}_{0} \qquad ( 12 )$$

where tHb_{b} is the total hemoglobin in the unit blood volume ΔV associated with Δt. K is the conversion coefficient from c_{tHb} to a PA signal, which is assumed as constant during the indicator dilution measurement. K accommodates systematic effects, such as a sensitivity of an ultrasound receiver or fluence in PA imaging. The term PA₀ represents the PA signal from all PA sources insensitive to the indicator concentration change. The amount of PA₀ out of PA_{b} can be conceptually quantified, without physically separating the PA sources into PA₀ and PA_{b} portions. At the measurement point of the arterial vessel, the total hemoglobin in tHb_{b} is decreased due to the added portion of the isotonic solution, ΔV_{I}(t). For this situation, the measured PA signal variation per Δt can be described as

$$PA ( t ) = K \cdot {c}_{tHb} ( t ) + {PA}_{0} = K \frac{{tHb}_{m} ( t )}{{V}_{m} ( t ) + {V}_{I} ( t ) ′} + {PA}_{0} \qquad ( 13 )$$

where ΔVₘ(t)+ΔV_{I}(t)=ΔV. Due to ΔV_{I}(t), the total hemoglobin in V, tHbₘ(t) is smaller than tHb_{b}. However, the hemoglobin concentration in pure blood (i.e., the blood without the isotonic solution) is not changed by the injection, so

$$\frac{{tHb}_{b}}{Δ V} = \frac{{tHb}_{m} ( t )}{Δ {V}_{m} ( t )} \qquad ( 14 )$$

By substituting Eq. (14) to Eq. (13), the measured PA signal, PA(t) is

$$PA ( t ) = \frac{K Δ {V}_{m} ( t ) \cdot {tHb}_{b}}{Δ {V}^{2}} = \frac{K [ Δ V - Δ {V}_{I} ( t ) ] \cdot {tHb}_{b}}{Δ {V}^{2}} . \qquad ( 15 )$$

Considering Eq. (12), Eq. (15) is further developed to

$$PA ( t ) = {PA}_{b} [ 1 - α \frac{{V}_{I} ( t )}{V} ] , \qquad ( 16 )$$

where α=(PA_{b}−PA₀)/PA_{b} and a is always less than 1, if PA₀ is not zero. Integrating both sides of Eq. (16) in time derives the blood flow rate as

$$F = α {V}_{It} / {\int }_{0}^{∞} [ 1 - \frac{PA ( t )}{{PA}_{b}} ] d t \qquad ( 17 )$$

where Eq. (11) is applied to the derivation of Eq. (17). In the process from Eq. (15) to (17), it is assumed that PA_{b} and PA₀ are constant during the indicator dilution measurement. Since a PA signal measured is decreased due to the isotonic injectate, the denominator of Eq. (17) indicates the area between PA dilution curve and the normalized baseline. The normalization in the integration of Eq. (17) is obtained during the derivation process, which is from the PA signal being proportional to the inverse of the amount of an isotonic concentration. Eq. (17) indicates that if there is some portion in the PA source, which is effectively insensitive to the indicator concentration in the blood vessel, the estimated hemodynamic parameter (e.g., cardiac output) is overestimated unless α is considered. Furthermore, the amount of overestimation is proportional to α, so the effect of α cannot be negligible. One method to possibly reduce the effect of α is to increase an incident photon power and decrease an ultrasound receiver's resolution so that the portion of PA₀ is getting smaller (α is close to 1). This approach is based on the assumption that the majority of PA₀ in α is generated from the hemoglobin molecules close to the vessel wall. Alternatively, the value or range of α for a given PA system can be determined from a specific clinical situation, which is the calibration factor for the hemodynamic parameter (e.g., cardiac output) in that clinical situation.

The disclosed embodiments are provided in the context of indicator dilution curves. However, it should be understood that the disclosed techniques may be applied to other PA monitoring systems. Further, while the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A monitor, comprising:
a memory storing instructions for:
receiving a signal from an acoustic detector configured to detect a photoacoustic effect from light emitted into a patient's tissue, wherein the signal is representative of an indicator dilution;
dividing the signal into a plurality of blocks, wherein each block includes a same number of epochs;
arranging the respective epochs within each block of the plurality of blocks in an ensemble arrangement;
identifying a region of each respective epoch within each block that corresponds to a blood vessel response;
calculating an indicator dilution curve based on the identified regions of the epochs;
identifying a signal region of the indicator dilution curve;
extracting one or more features from the signal region of the indicator dilution curve; and
determining a physiological parameter based on the one or more features; and
a processor configured to execute the instructions.

2. The monitor of claim 1, wherein the instructions comprise calculating an average signal for each block prior to identifying the region corresponding to the blood vessel response, and identifying the region corresponding to the blood vessel response for each average signal.

3. The monitor of claim 2, wherein the instructions for calculating the average signal comprise utilizing k-means clustering to divide the respective epochs within each block into clusters, aligning the epochs within each cluster, calculating a partial average for each cluster, calculating a peak-to-peak amplitude for each partial average, and selecting the partial average with the largest peak-to-peak amplitude as the average signal.

4. The monitor of claim 2, wherein the instructions for calculating the average signal comprise utilizing Woody filtering to reiteratively align the respective epochs within each block based on a current average signal template based on the respective epochs, calculate a current average signal template based on the aligned epochs, and calculate the mean square value between the current average signal template and a previous average signal template, wherein the current average signal template is used as the average signal if the mean square value is less than a threshold.

5. The monitor of claim 2, wherein the instructions for calculating the average signal comprise setting a temporally first epoch of the respective epochs of a block as an anchor epoch, correlating lags for each of the remaining epochs of the block to the anchor epoch, selecting a respective lag for each of the remaining epochs based on a correlation with anchor epoch, aligning the selected lags for each of the remaining epochs based on their respective correlation to the anchor epoch relative to the anchor epoch, and calculating the average signal based on the aligned epochs.

6. The monitor of claim 1, wherein the instructions comprise removing noise from the signal prior to calculating the indicator dilution curve, wherein removing noise from the signal comprises calculating wavelet coefficients for each epoch within each block, and performing an inverse transform to remove noise from the respective epochs in each block.

7. The monitor of claim 1, wherein the instructions for calculating the indicator dilution curve comprise utilizing complex demodulation to calculate an analytic signal for each epoch of each block, calculating an envelope based on the respective analytic signal, determining an area of each envelope within the identified region of each epoch, and utilizing each area as a sample on the indicator dilution curve.

8. The monitor of claim 1, wherein the instructions for calculating the indicator dilution curve comprise analyzing a power of each epoch to generate a sample on the indicator dilution curve.

9. The monitor of claim 1, wherein the instructions comprise removing noise from the indicator dilution curve prior to extracting the one or more features from the signal region of the indicator dilution curve by applying a selected filter to the indicator dilution curve, wherein the selected filter is selected via adaptive filter selection to determine a filter that generates the highest signal-to-noise ratio relative to other filters when applied to the indicator dilution curve.

10. The monitor of claim 1, wherein the instructions comprise removing noise from the indicator dilution curve prior to extracting the one or more features from the signal region of the indicator dilution curve by utilizing total variation minimization to filter the indicator dilution curve.

11. The monitor of claim 1, wherein the instructions comprise normalizing the signal region of the indicator dilution curve relative to a maximum or minimum value of the signal region to extracting the one or more features from the signal region.

12. The monitor of claim 1, wherein the instructions for identifying the signal region of the indicator dilution curve comprise low-pass filtering the indicator dilution curve, finding a minimum peak of the indicator dilution curve, and determining a beginning and an end of the signal region based on samples values of the indicator curve relative to a percentage of a minimum value before and after of the minimum peak, respectively.

13. The monitor of claim 1, wherein the one or more features comprise an area, an amplitude, or a duration of the signal region.

14. The monitor of claim 1, wherein the physiological parameter comprises cardiac output.

15. The monitor of claim 1, wherein the instructions for identifying a region of each respective epoch that corresponds to a blood vessel response comprise utilizing an ensemble-averaged envelope calculated for each block of the plurality of blocks based on the respective epochs within each block.

16. The monitor of claim 1, wherein the instructions for determining a physiological parameter based on the one or more features comprise utilizing ridge regression.

17. A method for determining a physiological parameter of a patient, comprising:
using a processor for:
receiving a signal from an acoustic detector configured to detect a photoacoustic effect from light emitted into a patient's tissue, wherein the signal is representative of an indicator dilution;
dividing the signal into a plurality of blocks, wherein each block includes a same number of epochs;
arranging the respective epochs within each block of the plurality of blocks in an ensemble arrangement;
identifying a region of each respective epoch that corresponds to a blood vessel response utilizing an ensemble-averaged envelope calculated for each block of the plurality of blocks based on the respective epochs within each block;
calculating an indicator dilution curve based on the identified regions of the epochs;
identifying a signal region of the indicator dilution curve;
extracting one or more features from the signal region of the indicator dilution curve; and
determining a physiological parameter based on the one or more features utilizing ridge regression.

18. The method of claim 17, comprising using the processor for calculating an average signal for each block prior to identifying the regions corresponding to the blood vessel response, and identifying the region corresponding to the blood vessel response for each average signal.

19. A non-transitory computer-readable medium having computer executable code stored thereon, the code comprising instructions for:
receiving a signal from an acoustic detector configured to detect a photoacoustic effect from light emitted into a patient's tissue, wherein the signal is representative of an indicator dilution;
dividing the signal into a plurality of blocks, wherein each block includes a same number of epochs;
identifying a region of each respective epoch within each block that corresponds to a blood vessel response;
calculating an indicator dilution curve based on the identified regions of the epochs;
identifying a signal region of the indicator dilution curve;
extracting one or more features from the signal region of the indicator dilution curve; and
determining a physiological parameter based on the one or more features utilizing ridge regression.

20. The non-transitory computer-readable medium of claim 19, wherein the code comprises instructions for arranging the respective epochs within each block of the plurality of blocks in an ensemble arrangement prior to identifying a region of each respective epoch within each block that corresponds to a blood vessel response.
